(19) United States
(12) Patent Application Publication
(10) Pub. No.: US 2025/0144682 A1
(43) Pub. Date: May 8, 2025

(54) SYSTEMS, DEVICES, AND METHODS FOR DEACTIVATING ALKALI METAL DEVICES

(71) Applicant: Candesant Biomedical, Inc., Nashville, TN (US)

(72) Inventors: Jesse N. ROSEN, Albany, CA (US); Michael J. SHAWVER, Petaluma, CA (US); Patrick H. RUANE, El Dorado Hills, CA (US); Niquette L. HUNT, Boca Raton, FL (US); Chad A. FOLLMAR, Lafayette, CO (US); Christopher SEIGHMAN, Denver, CO (US); Clayton COLE, Longmont, CO (US)

(21) Appl. No.: 18/749,486

(22) Filed: Jun. 20, 2024

Related U.S. Application Data
(60) Provisional application No. 63/509,496, filed on Jun. 21, 2023.

(51) Int. Cl. B09B 3/70; B09B 101/65

(52) U.S. Cl. CPC B09B 3/70; B09B 2101/65

(57) ABSTRACT

The present disclosure relates to systems, devices, and methods for deactivating an alkali metal device. In an embodiment, the present disclosure relates to a deactivation container comprising a container body defining a cavity configured to receive a device comprising an alkali metal, the cavity containing an alkali metal reactant or an alkali metal solubilizer, a lid coupled to the container body and partially received in the cavity in a closed position, the lid comprising a pressure relief mechanism to release gas from a top of the deactivation container during a deactivation process, and a support configured to maintain the deactivation container in an upright position during the deactivation process.

### CROSS REFERENCE TO RELATED APPLICATIONS

This application claims priority to U.S. Provisional Patent Application Ser. No. 63/509,496 filed Jun. 21, 2023, the contents of which are incorporated herein by reference in their entirety for all purposes.

### TECHNICAL FIELD

The present disclosure relates to systems, devices, and methods for deactivating a device with a reactive substance, for example, deactivating an alkali metal patch after use in a medical treatment.

### BACKGROUND

Devices for use in a medical treatment may include an amount of a reactive substance. The disposal of such devices may be hazardous due to a possibility of an inadvertent and undesirable chemical reaction between the reactive substance and other waste materials. The chemical reaction may result in hazardous byproducts, a significant increase in temperature, a significant increase in pressure, and other conditions that may create dangerous conditions for patients, medical personnel, and/or medical facilities. Thus, it may be desirable to deactivate the reactive substance to allow for easier and safer disposal of the devices. Accordingly, systems, devices, and methods for deactivating a medical device with a reactive substance, such as a medical device comprising an alkali metal, safely, effectively, and securely are desirable.

### SUMMARY

The present disclosure relates generally to systems, devices, kits, and methods for deactivating an alkali metal device. In general, the deactivation systems and kits described herein may be used with a device comprising an alkali metal and may assist in deactivating and/or disposing of the device. The deactivation systems and/or kits may include a deactivation container and a support extending from the deactivation container. The deactivation container may include a body configured to receive a device comprising an alkali metal. The deactivation container body may have an alkali metal reactant or an alkali metal solubilizer therein. The support may be configured to maintain the deactivation container in an upright position during a deactivation process. In some variations, the support may be releasably coupled to the deactivation container, while in other variations, the support may be integrally formed with the deactivation container. The support may be a base including a chamber configured to receive at least one deactivation container. The base may further include a bottom, a first pair of sidewalls, and a second pair of sidewalls defining the chamber. In some variations, a height of at least one sidewall of the first and second pairs of sidewalls may be less than a height of the deactivation container. In some variations, the first and second sidewalls of the first pair of sidewalls may be attached to opposite ends of the bottom and are parallel to one another, and the third and fourth sidewalls of the second pair of sidewalls may be attached to opposite ends of the bottom and are parallel to one another. Each of the sidewalls may be substantially planar. The base may further include a cover releasably coupled to the first and second pairs of sidewalls. Further, the base may include a standoff member within the chamber, where the standoff member is configured to maintain a gap between the body of the deactivation container and each sidewall of the base. The standoff member may have an opening configured to receive the deactivation container, and the opening may have a shape that corresponds to a shape of the deactivation container. In some variations, the standoff member may be coupled to an inner surface of at least one sidewall. Moreover, the deactivation container may be a first deactivation container, and the deactivation kit may further include a second deactivation container. The support may be a base comprising a chamber and a standoff member within the chamber, the standoff member configured to maintain gaps between each of the body of the first deactivation container, the body of the second deactivation container, and sidewalls of the base. In some variations, the standoff member may include a first opening configured to receive the first deactivation container and a second opening configured to receive the second deactivation container, where the first opening may have a shape that corresponds to a shape of the first deactivation container and the second opening may have a shape that corresponds to a shape of the second deactivation container. The deactivation container may include a lid coupled to the deactivation container body. In some variations, the lid may be a one-way valve. Furthermore, the alkali metal reactant or the alkali metal solubilizer may include a solution having one or more of ethanol, water, isopropanol, t-butanol, stearyl alcohol, tris(trimethylsilyl)methanol, and propylene glycol. The solution may further include an additive configured to maintain a pH measurement of the solution between about 2.5 and about 12.5. The additive may include one or more of citric acid, acetic acid, glycolic acid, azelaic acid, and salicylic acid. Moreover, the deactivation container may be a first deactivation container, the kit may further include a second deactivation container, and the support may be a bridge connecting the first and second deactivation containers. One or both of the first deactivation container and the second deactivation container may extend transversely through the bridge. Further, one or both of the first and second deactivation containers may extend from a bottom surface of the support past a top surface of the support. In some variations, the bridge may be coupled to a proximal portion of the deactivation container adjacent to an opening of a cavity defined by the body of the deactivation container. In some variations, the bridge may include one or more of a plate and a crossbeam that maintains a gap between the first and second deactivation containers. A top surface of the plate may be parallel to a plane formed through a longitudinal axis of the opening of the cavity. In some variations, the deactivation kit may further include a lid releasably received at least partially within a cavity defined by the body of the deactivation container. The lid may include a pressure management feature. In some variations, the pressure management feature may include a vent and a membrane covering the vent and configured to limit fluid exchange between the cavity of the deactivation container and an external environment. The vent may extend transversely through the lid.

A deactivation container may include a container body that defines a cavity therein. The cavity may be configured to receive the device and may contain an alkali metal reactant or an alkali metal solubilizer. The deactivation container may also include a lid coupled to the container body. The lid may be partially received within the cavity in a closed position. The lid may include a include a pressure management feature to release gas from a top of the deactivation container. The lid may include at least one indentation. Moreover, the lid may be coupled to the container body by a hinge. The hinge may be rotatably coupled to a bar of the container body. In some variations, the hinge may be coupled to a support coupled to the container body. The support may be integrally formed with or fixedly attached to the container body. The hinge may include a stop that prevents the lid from opening beyond a predetermined angle. The predetermined angle may be between about 90 degrees to about 180 degrees. The lid may include a latch. The alkali metal reactant or the alkali metal solubilizer may include a solution comprising one or more of ethanol, isopropanol, t-butanol, stearyl alcohol, tris(trimethylsilyl)methanol, and propylene glycol. The deactivation container may include an alignment feature within the cavity. Further, the pressure management feature may include a vent with a lumen extending transversely through the lid. In some variations, the lid may further include a recess on a bottom of the lid at least partially surrounding the vent, and a membrane within the recess and configured to limit fluid exchange between the cavity of the deactivation container and an external environment via the vent. A diameter of the recess may be greater than a diameter of the vent. In some variations, the diameter of the vent may be about 0.1 mm to about 2 mm. Moreover, the membrane may have a pressure rating of about 5 psi to about 8 psi. Furthermore, a thickness of the lid may increase from both of a first end and a second end of the lid to a central portion of the lid. The lid may further include a plurality of longitudinal ridges configured to increase a stiffness of the lid. The plurality of ridges may be on a top surface of the lid. The lid may further include one or more projections extending from a bottom surface of the lid and configured to contact the device within the deactivation container when the lid is in the closed position. Moreover, the pressure management feature may include a valve extending through the lid and at least partially into the cavity of the container body. The valve may be an umbrella valve.

A deactivation container may include a container body comprising a bottom, a top, and at least one sidewall. The top may include an opening to a cavity defined by the container body for receiving an alkali metal reactant or an alkali metal solubilizer therein. The deactivation container may include a lid coupled to the container body, and the lid may be movable between an open position and a closed position. In the closed position, a portion of the lid may traverse the opening and occupy a predetermined volume of the cavity. The portion may extend along a perimeter of the lid and/or a width of the lid. Additionally, the portion may define at least one channel configured to divert a fluid towards the pressure relief valve. Further, the portion may include a plurality of projections. In some variations, the predetermined volume may be at least 75% of the volume of the cavity. In some variations, a height of the at least one sidewall may be greater than a length and a width of the bottom. The lid may include a gasket extending along a perimeter of the lid. The gasket may be adjacent to the portion, and the gasket may be configured to engage an edge of at least one sidewall of the container body. In some variations, the security mechanism may be configured to increase a force applied to the gasket.

A deactivation container may include a container body comprising a bottom, a top opposite the bottom, and at least one sidewall therebetween. The container body may define a cavity for receiving a device comprising an alkali metal. The cavity may contain an alkali metal reactant or an alkali metal solubilizer. A lid may be coupled to the container body for releasably sealing the top of the deactivation container. The lid may include a one-way valve configured to release gas from the top of the deactivation container while a remainder of the top of the deactivation container remains sealed. In some variations, the lid may include a one-way valve configured to release gas from the top of the deactivation while a perimeter of the opening remains sealed.

A deactivation container may include a container body defining a cavity configured to receive an alkali metal device, a lid coupled to the container body and including a pressure relief valve, and a security mechanism releasably coupled to the lid configured to maintain the pressure relief valve in a closed configuration. After releasing the security mechanism, the pressure relief valve may be configured to open when a differential between a cavity pressure and an environmental pressure exceeds a predetermined threshold. The predetermined threshold may be between about 1 psi and about 10 psi. The security mechanism may include one or more of a clip, a latch, a magnet, a hook, an elastic band, and a cable tie. In some variations, the security mechanism may be a clip. The security mechanism may include a first section coupled to a first sidewall of a pair of sidewalls of the container body and a second section is coupled to a second sidewall of the pair of sidewalls of the container body. The security mechanism may include a first section separated from a second section by a first perforated portion, and a third section separated from the second section by a second perforated portion. In some variations, the second section may be separable from the first and third sections along the first and second perforated portions. Separating the second section from at least one of the first and third sections may disengage the security mechanism. In some variations, the first, second, and third sections may form a strip. The first and second perforated portions may have perforated lines across the strip.

A deactivation container may include an annular container body defining a cavity therein and configured to receive an alkali metal device, where the cavity may contain an alkali metal reactant or an alkali metal solubilizer therein. The deactivation container may also include a lid configured to engage the annular container body and to be received at least partially within the cavity of the annular container body in a closed position, and the lid may include a pressure management feature to release to release gas from a top of the deactivation container during a deactivation process. In some variations, the lid may have external threads configured to engage with internal threads of the cavity of the annular container body. In some variations, the pressure management feature may include a vent having a lumen traversing the lid and a membrane covering the vent.

Methods of deactivating a device with an alkali metal are also described herein. Generally, methods of deactivating a device including an alkali metal may comprise placing the device into a cavity of a deactivation container to contact the alkali metal with an alkali metal reactant or alkali metal solubilizer. The deactivation container may be in an upright position and may include a lid releasably sealing a top opening of the cavity. The lid may include a pressure relief valve. Methods may further include closing the lid to seal the top opening of the cavity. Gas generated during a reaction of the alkali metal and the alkali metal reactant or alkali metal solubilizer may vent through the pressure relief valve. Methods may also include inserting the deactivation container into a base prior to placing the device into the cavity. The device may be in the form of a patch. As such, the methods may further include folding the patch before placing the patch into the cavity. Additionally, or alternatively, the methods may further include placing a porous cover on the patch adhesive before folding the patch. In some variations, the lid may further include a latch configured to releasably engage a sidewall of the deactivation container. Closing the lid may further include engaging the latch with the sidewall. After placing the device into the cavity of the deactivation, the device may be retained in the cavity for a predetermined deactivation period. The predetermined deactivation period may be between about 30 minutes to about 8 hours. A sidewall temperature of the deactivation container may be maintained at less than about 50 degrees C. during the predetermined deactivation period. Methods may further include disengaging a security mechanism configured to maintain the pressure relief valve in a closed configuration from the pressure relief. In some variations, methods may include inserting the deactivation container into a biohazard waste bag. In some variations, the alkali metal reactant or the alkali metal solubilizer may include a solution comprising one or more of ethanol, water, isopropanol, t-butanol, stearyl alcohol, tris(trimethylsilyl)methanol, and propylene glycol. The the solution further comprises an additive configured to maintain a pH of the solution below a predetermined pH level during the predetermined deactivation period. In some variations, the additive may include one or more of citric acid, acetic acid, glycolic acid, azelaic acid, and salicylic acid.

The methods may comprise providing a deactivation container removably positioned within a base, the deactivation container including a container body defining a cavity holding an alkali metal reactant or an alkali metal solubilizer and a lid coupled to the container body. The lid may include a pressure relief valve and a security mechanism restraining the pressure relief valve. Methods may further include releasing the security mechanism from the pressure relief valve, placing the device into the cavity to contact the alkali metal with the alkali metal reactant or the alkali metal solubilizer, and allowing the device to remain in contact with the alkali metal reactant or the alkali metal solubilizer for a predetermined deactivation period.

Methods may comprise placing an alkali metal device in a deactivation container having a pressure relief valve in a lid thereof and a body defining a cavity with an alkali metal reactant or an alkali metal solubilizer. The deactivation container may be in an upright position. Methods may further include sealing the container such that gas vents through the pressure relief valve in the lid during the deactivation of the alkali metal device.

In some variations, one or more devices or components of the systems described herein may be packaged or may otherwise form a deactivation kit.

Additional embodiments, features, and advantages of the invention will be apparent from the following detailed description and through practice of the invention.

### BRIEF DESCRIPTION OF THE DRAWINGS

FIG. 1 is a partially exploded view of an illustrative variation of a deactivation kit.

FIG. 2 is an illustrative variation of a deactivation container in a closed configuration.

FIG. 3 is an illustrative variation of a deactivation container in an open configuration.

FIGS. 4A-4E illustrate a top view (FIG. 4A), a front view (FIG. 4B), a side view (FIG. 4C), a rear view (FIG. 4D), and a perspective view (FIG. 4E) of an illustrative variation of a deactivation container body.

FIGS. 5A-5F illustrate a first perspective view (FIG. 5A), a second perspective view (FIG. 5B), a top view (FIG. 5C), a bottom view (FIG. 5D), a front view (FIG. 5E), and a side view (FIG. 5F) of a lid for a deactivation container.

FIGS. 6A-6F illustrate a first perspective view (FIG. 6A), a second perspective view (FIG. 6B), a top view (FIG. 6C), a bottom view (FIG. 6D), a front view (FIG. 6E), and a side view (FIG. 6F) of another variation of a lid of a deactivation container.

FIGS. 7A-7C illustrate a front view (FIG. 7A), a side view (FIG. 7B), and a perspective view (FIG. 7C) of a variation of a gasket of a deactivation container.

FIGS. 8A-8C illustrate a perspective view (FIG. 8A), a side view (FIG. 8B), and a top view (FIG. 8C) of an illustrative variation of a pressure relief valve of a deactivation container.

FIG. 9 is a cross-sectional view of an illustrative variation of a pressure relief valve for a deactivation container.

FIGS. 10A-10D illustrates a first side view (FIG. 10A), a second side view (FIG. 10B), a first perspective view (FIG. 10C), and a second perspective view (FIG. 10D) of an illustrative variation of a security mechanism of a lid of a deactivation container.

FIGS. 11A-11C illustrate a first side view (FIG. 11A), a first perspective view (FIG. 11B), and a second perspective view (FIG. 11C) of an alternative variation of a security mechanism of a lid of a deactivation container.

FIG. 12 shows a perspective view of an illustrative variation of a base of a deactivation kit.

FIGS. 13A-13B show a first perspective view (FIG. 13A) and a second perspective view (FIG. 13B) of another illustrative variation of a base of a deactivation kit.

FIG. 14 shows a perspective view of yet another illustrative variation of a base of a deactivation kit with deactivation containers releasably coupled thereto.

FIG. 15 shows a cross-sectional view of a further illustrative variation of a base of a deactivation kit with a deactivation container releasably coupled thereto.

FIG. 16 shows a cross-sectional view of yet another illustrative variation of a base of a deactivation kit with a deactivation container releasably coupled thereto.

FIGS. 17A-17C show a top view (FIG. 17A), a first side view (FIG. 17B), and a second side view (FIG. 17C) of an alkali metal device comprising a patch.

FIG. 18A and FIG. 18B are flowcharts of illustrative variations of a deactivation method for an alkali metal device.

FIG. 19 shows an exploded view of another illustrative variation of a deactivation kit.

FIG. 20 shows a perspective view of an illustrative variation of a deactivation kit in a closed configuration.

FIGS. 21A-21B show a perspective view (FIG. 21A) and a cross-sectional view (FIG. 21B) of another illustrative variation of a deactivation container.

FIGS. 22A-22C show a top view (FIG. 22A), a side view (FIG. 22B), and a bottom view (FIG. 22C) of another illustrative variation of a lid of a deactivation container.

FIGS. 23A-23B show a perspective view (FIG. 23A) and a top view (FIG. 23B) of an illustrative variation of a deactivation container having a bridge coupled thereto.

### DETAILED DESCRIPTION

Disclosed herein are devices, systems, and methods for deactivating a device comprising a reactive material, such as an alkali metal (e.g., lithium, sodium, potassium, rubidium, cesium, francium), an oxide thereof (e.g., sodium oxide, potassium oxide), or an alloy thereof, after use of the device, such as in a method of treatment. Devices comprising a reactive material may have a variety of medical and non-medical uses. For example, alkali metal devices may be used in the treatment of various medical conditions, such as, for example, skin wounds, rashes, hyperhidrosis (e.g., excessive sweating), limiting undesirable bacteria growth, odor reduction, or other medical or cosmetic conditions. In these instances, the alkali metal device may be configured to be applied to a treatment site on a human body, resulting in a chemical reaction between the alkali metal and a material in or on the body, such as, for example, water (e.g., in sweat) on and/or within the tissue at a treatment site. The resulting chemical reaction may generate energy (e.g., heat) upon contact between the alkali metal and water. The heat generated by the interaction of the alkali metal of the devices described herein and the water at the treatment site may provide a medical or cosmetic benefit and, in some variations, may sterilize or render aseptic a surface of skin, tissue, or in variations in which the treatment site is not on a human body, medical instrument(s) or other surfaces such as, a clinical work bench. Accordingly, the devices may be used in a clinical environment (e.g., a hospital, clinic, doctor's office), in a laboratory environment, or in a home environment. The device may be in the form of a patch that is intended to be single-use and disposable. In many instances, the alkali metal of the device may not be entirely consumed during the treatment so excess (i.e., unreacted) alkali metal may remain on the device such that a portion of the alkali metal remains capable of reacting with water. In these instances, the remaining or excess alkali metal may create challenges to disposal of the device in a typical manner, as the remaining or excess alkali metal may readily and inadvertently react with water in an unintended manner (e.g., not at a treatment site and/or within an appropriate clinical or home environment), creating potentially hazardous conditions. Therefore, it is desirable to deactivate the remaining alkali metal to avoid this potentially harmful reaction after the alkali metal device has been used for treatment.

Accordingly, described herein are devices, systems, and methods for deactivating an alkali metal device after use. More specifically, after the alkali metal device has been used, the alkali metal of the alkali metal device may be deactivated in a deactivation process that may occur in a deactivation container. The alkali metal device may then be disposed of within, or independently of, the deactivation container. The deactivation container may define a cavity configured to receive the device and the cavity may contain an alkali metal reactant or alkali metal solubilizer. The alkali metal reactant or alkali metal solubilizer may exothermically react with the alkali metal of the alkali metal device. This exothermic reaction may increase the temperature of the alkali metal reactant or solubilizer, and in some instances, of at least a portion of the deactivation container itself. The reaction between the alkali metal and the alkali metal reactant or solubilizer may also result in a gaseous byproduct, which may increase the pressure in the deactivation container, such as, for example, in variations in which the deactivation container is closed (e.g., sealed) during the deactivation process. While the deactivation process may result in an increased temperature of and/or increased pressure in the deactivation container, the deactivation container may be configured to safely contain the deactivation process such that the deactivation container remains safe to handle (e.g., touch, hold, move) during the deactivation process. For example, the deactivation container may comprise a lid configured to safely release fluid, such as a gaseous byproduct of the alkali metal reaction. The lid may release the fluid via a pressure management feature configured to release the gaseous byproduct therethrough. For example, the pressure management feature may control fluid exchange between a cavity defined by the deactivation container body and an external environment. In some variations, the pressure management feature may comprise a membrane vent. In some variations, the pressure management feature may comprise a pressure relief valve. Additionally, or alternatively, the deactivation container may be configured to maintain a predetermined position, such as an upright position, during the alkali metal reaction. The upright position may allow for the alkali metal reactant or solubilizer to surround the alkali metal device such that substantially all of the alkali metal of the alkali metal device contacts, and thereby reacts with, the alkali metal reactant or solubilizer. The deactivation container may be configured to maintain the temperature of external surfaces of the container at or below a threshold that is safe for human handling, and the upright position of the deactivation container may facilitate fluid flow that may spread and/or dissipate heat generated by the reaction. The upright position of the deactivation container may ensure safe release of the gaseous byproduct through the pressure relief valve without spilling or leaking the alkali metal reactant or solubilizer. In a further example, the deactivation container may be part of a deactivation system or kit including a base configured to receive the deactivation container. In these variations, the base may securely maintain, or may assist in securely maintaining, the upright position of the deactivation container before, during, and/or after the deactivation process.

### I. Deactivation Systems or Kits

Generally, deactivation kits for deactivating an alkali metal device may be configured to provide for safe deactivation and, thus, safe disposal, of the alkali metal device. Deactivation kits may comprise one or more deactivation containers each configured to receive one or more devices. The device may require deactivation such that any potential reactants of the device are rendered unreactive. For example, a device may comprise an alkali metal (e.g., a layer of an alkali metal or oxide thereof) that may exothermically react upon contact with water. Proper disposal of the device may be hazardous for the user without adequate deactivation beforehand of any unreacted alkali metal of the device. Accordingly, an exemplary deactivation kit may comprise a volume of an alkali metal reactant or solubilizer configured to react with the alkali metal of the alkali metal device to inhibit any subsequent reaction between the alkali metal and water.

In an exemplary variation, a deactivation kit may comprise a support configured to maintain the container in an upright position and/or stabilize the container. The support may extend from the container such that the support may increase one or more dimensions of at least a portion of the container. For example, in some variations, the support may be a device that receives the container at least partially therein, thereby maintaining the container in an upright position and/or increasing the stability of the container when positioned in an upright position. Additionally, or alternatively, the support may include one or more structures that extend laterally outward from a portion of the container (e.g., a side portion adjacent the bottom, at the middle, and/or adjacent the top) thereby increasing a length and/or a width of the container in at least the portions to which the support is coupled. In some variations, the support may be releasably coupled to the container, while in other variations, the support may be integrally formed with the container body. In some variations, the support may fixedly couple together a plurality of deactivation containers (e.g., two or more deactivation containers). In some variations, the support may be configured to store or otherwise contain a deactivation container before, during and/or after a deactivation process.

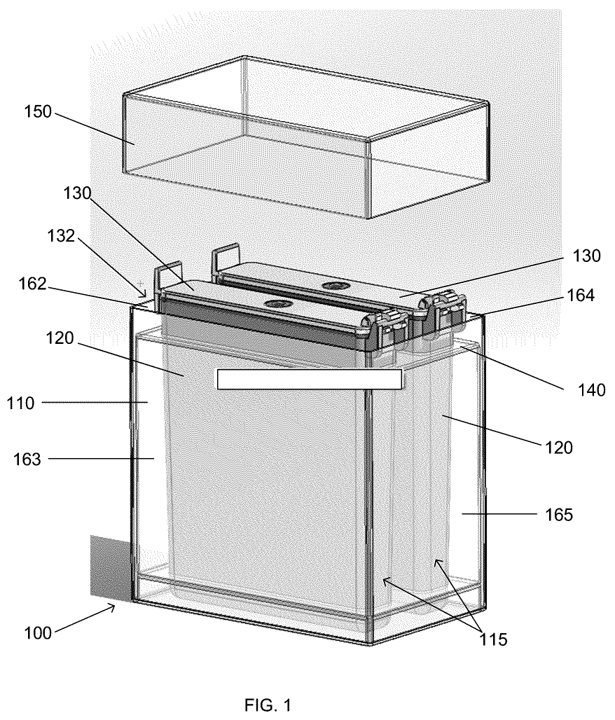

As will be described in detail herein, the deactivation container may couple to or be fixedly attached to a support, which may comprise a base and/or a bridge. FIG. 1 shows a partially exploded view of another illustrative variation of a deactivation kit (or system) 100 having a support that includes a base 110. The base 110, which in the variation depicted in FIG. 1, may be configured to receive a deactivation container 115. For example, as shown there, the base 110 may comprise sidewalls (e.g., 4), such as a first sidewall 162, a second sidewall 163, a third sidewall 164, and a fourth sidewall 165, that together define a top opening 132, configured to receive a deactivation container therethrough, and a cavity configured to receive a deactivation container 115 therein. In some variations, the base 110 may be configured to receive a plurality (e.g., two, three, four, or more) of deactivation containers 115. The base 110 may further comprise a bottom and one or more standoff members 140. The standoff members 140 may extend across the base 110 from a first inner surface of the base 110 to a second inner surface of the base 110, such as between the sidewalls. The standoff member 140 may be integrally formed with one or more of the sidewalls, or the standoff member 140 may be formed separately and attached to the sidewalls, e.g., releasably or non-releasably. The standoff member 140 may be partially or completely contained within the cavity of the base 110. As shown, the sidewalls 162, 163, 164, 165 may, in some variations, extend vertically beyond the standoff member 140. The standoff member 140 may be configured to maintain the deactivation container 115 in a predetermined position such as, for example, in an upright position. The standoff member 140 may be configured to maintain a gap between the deactivation container 115 and a sidewall of the base 110. The gap may be configured to provide a volume of air sufficient to reduce heat transfer between the deactivation container 115 and the base 110. In this way, the standoff member 140 may assist in maintaining each sidewall of the base 110 at a temperature safe for human handling during and/or after the deactivation process. The standoff member 140 and the opening of the base may each be configured to receive one or more deactivation containers 115. While described above as comprising four sidewalls and a bottom, as will be described in more detail herein, the base 110 need not. For example, in some variations, the base may only comprise two sidewalls and/or may comprise an open bottom.

In some variations, the deactivation kit 100 may further comprise a support lid 150. The support lid 150 may be placed over the opening of the base 110 such that the deactivation container 115 is enclosed within the base 110 and the support lid 150. The support lid 150 may be slidably received by the base 110. In some variations, the support lid 150 may be releasably coupled to the base 110 via a friction force, a clip, a latch, an adhesive, or by any other suitable means.

As mentioned above, the deactivation container 115 may be configured to receive a device, such as an alkali metal device. The device may be received within a body 120 of the deactivation container 115. The body 120 may define an opening and a cavity therein (not shown) shaped and sized to receive the device. In some variations, the opening and the cavity of the body 120 may comprise a shape (e.g., a cross-sectional shape) that is similar to the cross-sectional shape of the device (e.g., both have a square or rectangular cross-sectional shape) The opening of the deactivation container 115 may be covered by a lid 130. The lid 130 may fluidically seal the opening before, during, and/or after the deactivation process when the lid 130 is in a closed position (e.g., at least a portion of the lid being coplanar with the opening). The lid 130 may be releasably coupled to the deactivation container 115 via a hinge and/or a latch. In this way, the lid 130 may open and close as necessary to insert and remove the device from the cavity of the deactivation container 115.

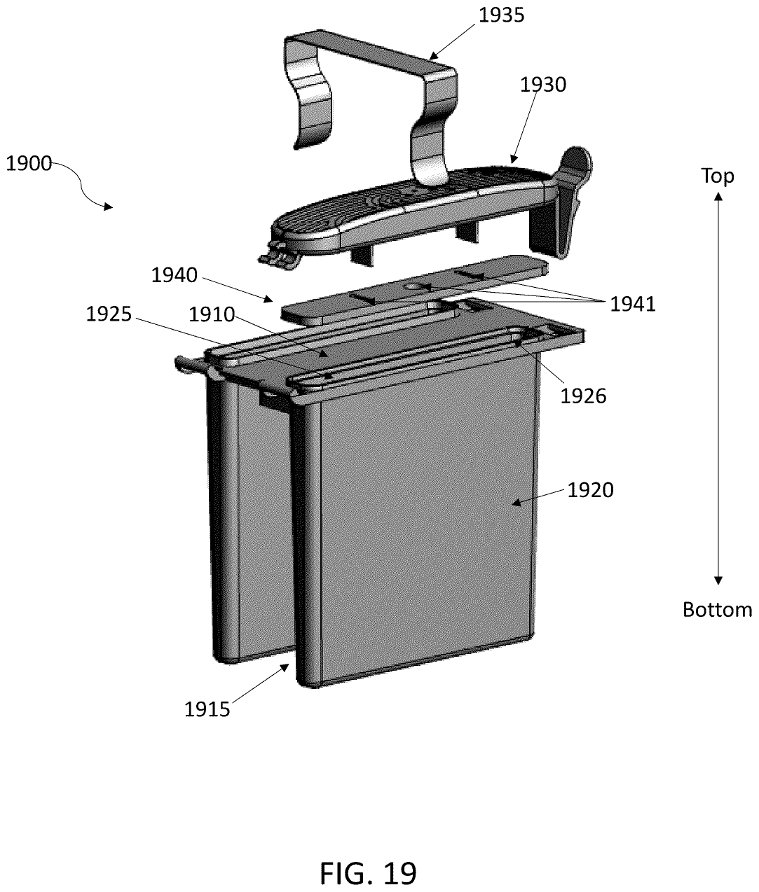

Moreover, FIG. 19 shows an exploded view of an illustrative variation of a deactivation kit (or system) 1900 having a support that includes a bridge 1910. Generally, the features shown and described with respect to FIG. 19 may be similar to the features shown and described with reference to FIG. 1 above. The bridge 1910 may be configured to receive the deactivation container 1915 in an opening therein or may be integrally formed with the deactivation container 1915. For example, the bridge 1910 may be a crossbeam or a plate that is attached or otherwise coupled to an first sidewall or surface of a body 1920 of the container. As shown, the bridge 1910 may be coupled to a first (e.g., top or proximal) portion of the container body 1920 to maintain the deactivation container 1915 in a predetermined (e.g., upright) position. A bottom or distal portion of the deactivation container 1915 may act as a leg to maintain the deactivation kit 1900 in the predetermined position. In some variations, the bridge 1910 may be part of a frame that at least partially surrounds an external perimeter of the deactivation container 1915.

As noted above, the deactivation container 1915 may be configured to receive a device, such as an alkali metal device. The device may be received within the container body 1920. For example, the container body 1920 may define a cavity 1925 shaped and sized to receive the device. In some variations, an opening 1926 of the cavity 1925 may comprise a shape (e.g., a cross-sectional shape) that is similar to the cross-sectional shape of the device (e.g., both may have a square or rectangular cross-sectional shape).

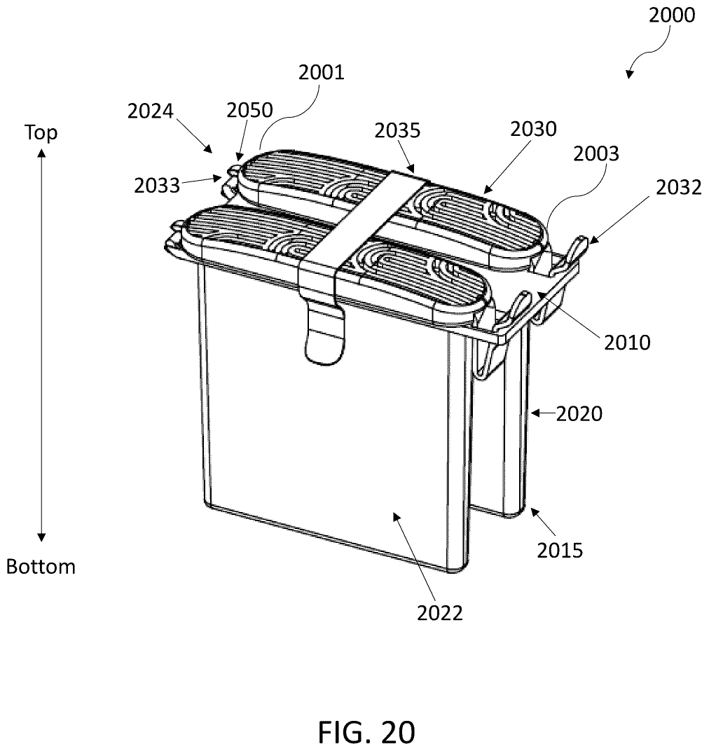

In some variations, the opening 1926 may be covered by a lid 1930. The lid 1930 may fluidically seal the opening 1926 before, during, and/or after a deactivation process when the lid 1930 is in a closed position (e.g., at least a portion of the lid being coplanar with the opening and/or a least a portion of the lid traversing the opening, as shown in FIG. 19 and FIG. 20). To do so, the lid 1930 may be configured to be at least partially received within the cavity 1925 of the deactivation container 1915. Additionally, or alternatively, the lid 1930 may be configured to sit atop a support extending from the deactivation container 1915, such as the bridge 1910, to seal the container 1915. For example, as mentioned above, the bridge 1910 may be part of a frame (e.g., a planar frame) that is configured to support at least a portion of a bottom perimeter of the lid 1930 thereon. Moreover, the lid 1930 may be releasably coupled to the support 1910 and/or to the deactivation container 1915 via a coupling mechanism 1924, which may be one or more of a hinge, a latch, a friction force, a clip, an adhesive, and any other suitable means. In this way, the lid 1930 may open and close as necessary to insert the device from the cavity 1925. Additionally, or alternatively, the lid 1930 may be releasably coupled to a support coupled to and extending from the deactivation container 1915, such as to the bridge 1910.

In some variations, the lid may include a pressure management feature 1970 and a security mechanism 1935 releasably coupled to the lid 1930 configured to maintain the pressure management feature 1970 in an obstructed configuration. The pressure management feature 1970 may be configured to manage a pressure within the deactivation container 1915 (e.g., within the cavity 1925) by, for example, preventing a buildup of pressure therein. In some variations, the pressure management feature 1970 may be configured to limit fluid exchange between the cavity 1925 and an external environment. As an example, FIG. 20 shows security mechanism 2035 releasably coupled to a pressure management feature (not shown) of the lid 2030. The security mechanism 2035 may be include one or more of a clip, a band, and an adhesive tape.

Further, in some variations, a deactivation kit may include one or more fluidic sealing components. For example, turning back to FIG. 19, the lid 1930 may include a gasket 1940. The gasket 1940 may be configured to couple to the lid 1930, such as to a bottom of the lid 1930. In some variations, the gasket 1940 may extend along a perimeter of the bottom of the lid 1930. In this way, the gasket 1940 may be adjacent to a portion of the lid 1930 such that the gasket 1940 engages an edge of at least one sidewall of the container body 1920. The gasket 1940 may include openings 1941 that are sized and shaped to receive or expose features on the bottom of the lid 1930. For example, a first opening of the gasket 1940 may be configured to expose a pressure management feature (e.g., a membrane vent) of the lid, which will be explained in more detail herein. As another example, one or more second openings (e.g., two second openings) of the gasket 1940 may be configured to receive a projection extending from the bottom of the lid 1930 so that the projection may maintain a device within the fluid of the cavity 1925, as is also described in more detail herein below.

### a. Deactivation Container

The deactivation kit described herein may comprise one or more (e.g., two, three, four, or more) deactivation containers. The deactivation container may be configured to provide a safe environment for deactivation of an alkali metal device while withstanding the chemical reaction therein without degrading, burning, melting, or otherwise corroding. The deactivation container may comprise a body that defines a cavity. The cavity may be configured to receive one or more devices with a reactant, such as an alkali metal device. The cavity may be further configured to contain a fluid such as, for example, an alkali metal reactant or alkali metal solubilizer, configured to react with alkali metal of an alkali metal device. In this way, a chemical reaction may safely occur between the alkali metal of the alkali metal device and the alkali metal reactant or solubilizer upon contact (e.g., during immersion in) of the alkali metal of the alkali metal device and the alkali metal reactant or solubilizer such that the deactivation container protects a user from byproducts of the chemical reaction such as, for example, heat, potentially hazardous gases, or flashes of light.

The body may comprise one or more walls in an arrangement suitable to receive one or more devices and/or one or more fluids. For example, the body may comprise a plurality of sidewalls and a bottom that together form any shape suitable for receiving an alkali metal device, such as, for example, a cube, a pyramid, a rectangular prism, or the like. In some variations, the body may comprise four sidewalls and may have a rectangular prism shape. The first and second sidewalls may be attached to opposite ends of the bottom. For example, the first and second sidewalls may be parallel to one another. In some variations, one or more of the sidewalls (e.g., all of the sidewalls) may be substantially planar. Additionally, or alternatively, one or more of the sidewalls may be partially or completely curved. For example, the body may include a combination of planar and curved sidewalls. Alternatively, in some variations, the body may be annular, forming a cylinder, a sphere, or other round shape.

The cavity may further comprise one or more alignment features extending from one or more sidewalls and/or the bottom of the cavity that are configured to maintain a gap between a device and a wall or the bottom of the deactivation container. The gap may reduce transfer of heat, which may be generated by the chemical reaction, to the walls and/or bottom of the deactivation container so that a user may still hold, pick up, or touch the external surfaces of the deactivation container. The gap may also allow a volume of fluid to circulate between the device and the wall of the container to reduce the temperature of the device during the reaction between the alkali metal and the reactant. The one or more alignment features may comprise a protrusion, an extension, a ridge, or a wall. In this way, the alignment features may be configured to maintain a gap between the device (i.e., the location of the chemical reaction) and the walls and/or bottom of the deactivation container. In some variations, the alignment features may also increase a structural rigidity parameter associated with at least a portion of the body such as stiffness, rigidity, tensile load rating, compressive load rating, or combination thereof.

The cavity may be further configured to receive a plurality of devices. The cavity may comprise two or more compartments. A compartment may be defined by a compartment divider, such as a wall within the cavity that extends from a bottom of the cavity to a top of cavity (i.e., ending coplanar with the opening). In this way, multiple fluids may be contained in each container such that the fluids are kept separate from one another within the same deactivation container body. In some variations, one or more compartments may be devoid of a fluid, such that a device may be inserted therein without reacting the device with a fluid.

The cavity of the body may be configured to contain a fluid configured to interact with a device. For example, the fluid may be ethanol, water, isopropanol, t-butanol, stearyl alcohol, tris(trimethylsilyl)methanol, propylene glycol, or a mixture of any of the above. The fluid may be in a gaseous state, a liquid state, or combination thereof. Accordingly, the body may comprise a material capable of safely containing the fluid described herein, such as a plastic (e.g., polypropylene, polycarbonate, polystyrene, polyethylene), a metal (e.g., stainless steel, steel, aluminum, copper), or glass.

The sidewalls may have a height and a width selected to allow for insertion of one or more devices therein, a sufficient cavity volume to allow the device to be substantially immersed in a fluid contained within the cavity (which may contribute to more even heat dissipation from a reaction occurring therein), maintain stability in an upright position of the deactivation container, and/or optimize packaging of one or more deactivation containers during transit to or from a clinical and/or home environment. In some variations, the height of each of the first and second sidewalls may be about 1 inch to about 6 inches (including all values and sub-ranges therein). For example, the height of each of the first and second sidewalls of may be about 1 inch to about 2 inches, about 1 inch to about 3 inches, about 1 inch to about 4 inches, or about 1 inch to about 5 inches. Additionally, or alternatively, the width of each of the first and second sidewalls may be about 1 inch to about 5 inches (including all values and sub-ranges therein). For example, the width of each of the first and second sidewalls width of each of the first and second sidewalls may be about 1 inch to about 2 inches, about 1 inch to about 3 inches, or about 1 inch to about 4 inches. In an exemplary variation, the height of each of the first and second sidewalls may be about 4.5 inches and the width of the first and second sidewalls may be about 3.9 inches. In some variations, the ratio of the height of the first and/or second sidewall to the width of the first and/or second sidewall may be about 1:1 to about 2:1 (including all values and sub-ranges therein). For example, the ratio of the height of the first and/or second sidewall to the width of the first and/or second sidewall may be about 1:1, about 1.1:1, about 1.2:1, about 1.3:1, about 1.4:1, about 1.5:1, about 1.6:1, about 1.7:1, about 1.8:1, about 1.9:1, or about 2:1.

The second pair of sidewalls may comprise a third and a fourth sidewall. In some variations, the height of each of the third and fourth sidewalls of the first pair may be about 1 inch to about 5 inches, about 2 inches to about 5 inches, about 3 inches to about 5 inches, or about 3 inches to about 6 inches. In some variations, the width of each of the third and fourth sidewalls of the first pair may be about 0.5 inches to about 1 inch, about 0.5 inches to about 2 inches, about 0.5 inches to about 3 inches, about 0.5 inches to about 4 inches, about 0.5 inches to about 5 inches, or about 0.5 inches to about 6 inches. The ratio of the height to the width of the third and fourth sidewalls of the second pair may be about 1:1, about 2:1, about 3:1, about 4:1, about 5:1, about 6:1, or about 7:1. The third and fourth sidewalls may be attached to opposite ends of the bottom. The third and fourth sidewalls may be parallel to one another.

The dimensions (e.g., length and/or width) of the first pair of sidewalls may be different than the dimensions of the second pair of sidewalls. For example, the width of each of the first and second sidewalls may be greater than a width of each of the third and fourth sidewalls. The ratio of the width of the first and/or second sidewall to the width of the third and/or fourth sidewall may be about 1:1 to about 7:1. For example, the ratio may be about 1:1, about 2:1, about 3:1, about 4:1, about 5:1, about 6:1, or about 7:1. In an exemplary variation, this ratio may be 4.4:1. The height of at least one sidewall of the first and second pairs may be greater than a height of a support, such as a base, of the deactivation kit. In an exemplary variation, the ratio of a container body sidewall height to a support (e.g., base) sidewall height may be about 0.5:1 to about 5:1. In some variations, the ratio may be about 0.6:1, about 0.7:1, about 0.8:1, about 0.9:1, about 1:1, about 1.1:1, about 1.2:1, about 1.3:1, about 1.4:1, about 1.5:1, about 1.6:1, about 1.7:1, about 1.8:1, about 1.9:1, or about 2:1.

As mentioned above, the body may be configured to receive and/or contain a fluid, such as an alkali metal reactant or an alkali metal solubilizer therein. In particular, the cavity of the body may be configured to contain a volume of fluid sufficient to react with (e.g., consume) all of the remaining alkali metal of the alkali metal device, while maintaining a safe temperature and pressure during the deactivation process. For example, the cavity may be configured to receive about 0.01 L to about 2 L of fluid, 0.05 L to about 0.25 L, or about 0.1 L to about 0.2 L In an exemplary variation, the cavity may be configured to receive about 0.115 L of fluid. In other variations, the cavity may be configured to receive about 0.1 L, about 0.15 L, about 0.2 L, about 0.3 L, about 0.4 L, about 0.5 L, about 1 L, about 1.5 L, about 2 L, or greater. In some variations, the cavity may be configured to receive no more than about 0.12 L, about 0.015 L, about 1 L, or about 2 L of fluid.

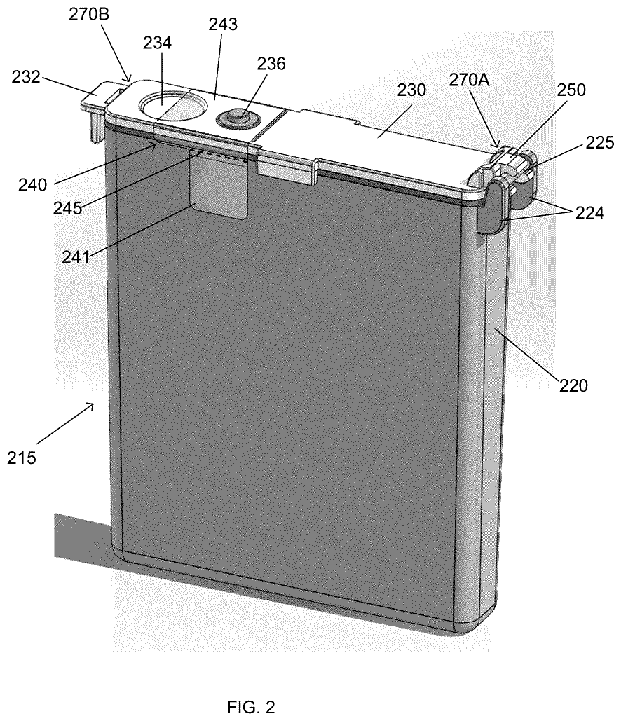

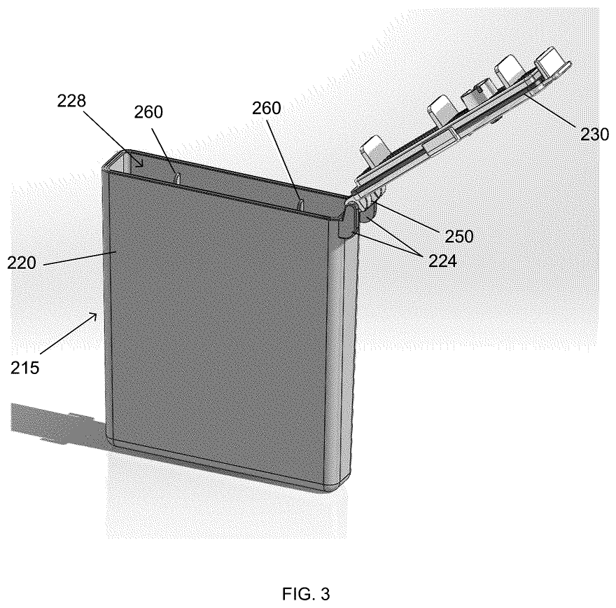

Referring to FIGS. 2 and 3, an exemplary variation of a deactivation container 215 is shown with a lid in an open position (FIG. 2) and in a closed position (FIG. 3). The open position of the lid 230 may reveal an opening 228 of the body 220. The opening 228 may be defined by an edge of one or more sidewalls of the body. The opening 228 may be configured to receive a device such as, for example, an alkali metal device therethrough. In some variations, the shape of the opening 228 may correspond to a shape of one or more devices, but need not as long as the one or more devices may be received through the opening. The shape of the opening 228 may also be sized such that a user may insert a finger or a hand through the opening 228 and into the cavity of the deactivation container. In this way, the user may remove the one or more devices, clean a surface of the cavity, and/or perform repair work of the deactivation container. In some variations, the deactivation container may be disposable.

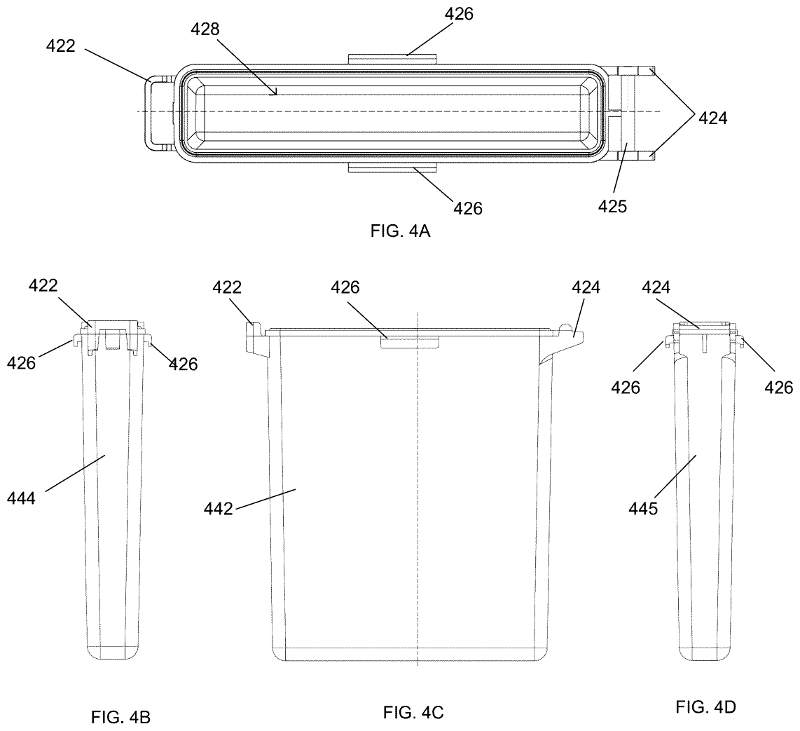

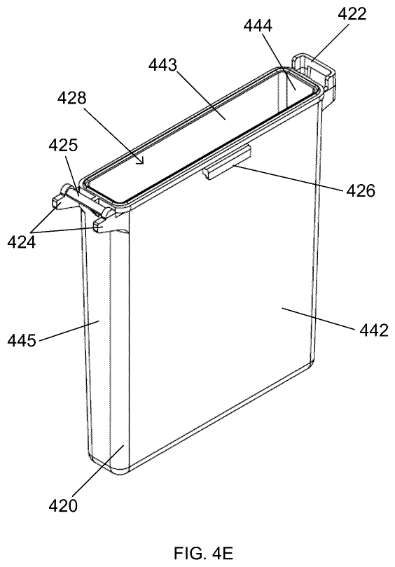

An exemplary variation of a body 420 is shown in FIGS. 4A-E. Similar to the variations described above, the body 420 may comprise a cavity configured to receive a device, such as an alkali metal device, and/or a fluid, such as an alkali metal reactant or an alkali metal solubilizer. The cavity may be defined by a bottom, a first pair of sidewalls, and a second pair of sidewalls of the body 420. In some variations, such as the variation shown in FIGS. 4A-4E, each sidewall of the body 420 may be substantially planar, while in other variations, one or more of the sidewalls of the body 420 may be curved (e.g., concave, convex). The first pair of sidewalls may comprise a first sidewall 442 opposite from a second sidewall 443 and the second pair of sidewalls may comprise a third sidewall 444 opposite from a fourth sidewall 445. The first sidewall 442 may be substantially parallel to the second sidewall 443 and the third sidewall 444 may be substantially parallel to the fourth sidewall 445. Each of the sidewalls may extend from the bottom of the container 420 to a top edge, and the top edges of the sidewalls may define the opening 428 of the container 420.

In some variations, the body may comprise one or more features configured to engage with the lid. In an exemplary variation, the body 420 may have a hinge 424 connected to a rod 425, where the rod 425 is configured to interact with clip on the lid to allow movement (e.g., rotation) of the lid relative to the body 420. In some variations, the rod 425 may be at a first end (defined by the edge of sidewall 445) of an opening 428 of the body 420 and the latch receiver 422 may be at a second end (defined by the edge of sidewall 444) opposite the first end of the opening 428. The rod 425 may be configured to receive a hinge clip of a lid (not shown). The rod 425 may comprise a protrusion, a cylinder, or other structure configured to receive the hinge clip of the lid. The rod 425 may be coupled to the container body via one or more standoffs 424 defined by the hinge 424, such that the rod 425 is spaced apart from the body 420. Thus, the rod 425 may not cover any portion of the opening of the body and may provide a pivot point around which the lid may rotate that allows for the lid to be received on and/or in the opening and to seat on the opening. In some variations, two standoffs may be used and each standoff may align with one of the first sidewall 442 and second sidewall 443, such that the rod 425 may extend along the entire width of the body. In other variations, the standoffs may be inset from the first sidewall 442 and second sidewall 443, such that the rod 425 may extend along only a portion of the width of the body. In such a variation, the rod 425 may be centered with respect to the width of the body.

In some variations, the body may further comprise a latch receiver 422 configured to interact with a latch on the lid to facilitate closure of the container and containment of the fluid and/or device therein. The latch receiver 422 may comprise a protrusion, a ledge, or an extension, which may have a shape (such as an indentation or depression) corresponding to the latch on the lid. In this way, the latch receiver 422 may have sufficient elasticity to releasably receive the latch of the lid. The latch receiver 422 may be coupled to the sidewall 444 of the body 420 so that the lid may be retained securely in a closed position while completely covering the opening 428. Accordingly, the latch receiver may extend outwards from the sidewall 444 and thus not cover any part of the opening 428. Additionally, the latch receiver 422 may be centered along, and extend the entire width of, the sidewall 444.

The body may further comprise one or more features configured to enable a user to easily grasp, pick up, and/or move the body. For example, an exothermic reaction may occur within the body 420 that increases a temperature of a body sidewall, but may not increase a temperature of an extension 426 to an equivalent temperature. The extension 426 may comprise a surface configured to pick up and/or carry the deactivation container, such as one or more of a ledge, a protrusion, a ridge, an overhang, and a hook, and an indentation. For example, a user may apply a finger to a extension 426 such that the user may lift up the body 420. In this way, the extension element may facilitate portability of the body during and/or after an exothermic reaction therein. In an exemplary variation, the body 420 may further comprise two or more extension 426. The extension 426 may be positioned on, and in some variations, extend from, an external surface of the first sidewall 442 and second sidewall 443 of the body 420 near or adjacent the opening 428. The extension 426 may be positioned so that the deactivation container may be picked up without spilling any fluid contained therein. The extension 426 may extend in a direction different than each of the hinge 424 and latch receiver 422. In further variations, the hinge 424 and latch receiver 422 may be used to carry the container, similarly as described above with respect to extension 426. Additionally, or alternatively, in some variations, the extension 426 may be a hold down feature for the security mechanism (e.g., clip). For example, the extension 426 may be configured to engage the security mechanism to maintain its position on the lid.

The ledge 426 may comprise a width and a length suitable to receiver a user's finger. In some variations, the engagement protrusion width may be about 0.01 inches to about 0.2 inches, about 0.05 inches to about 0.2 inches, about 0.05 inches to about 0.15 inches, about 0.07 inches to about 0.11 inches or about 0.085 inches to about 0.095 inches. In an exemplary variation, the engagement protrusion width may be about 0.09 inches. In some variations, the engagement protrusion length may be about 0.25 inches to about 6 inches, about 0.5 inches to about 2 inches, about 0.5 inches to about 1 inch, or about 0.65 inches to about 0.85 inches.

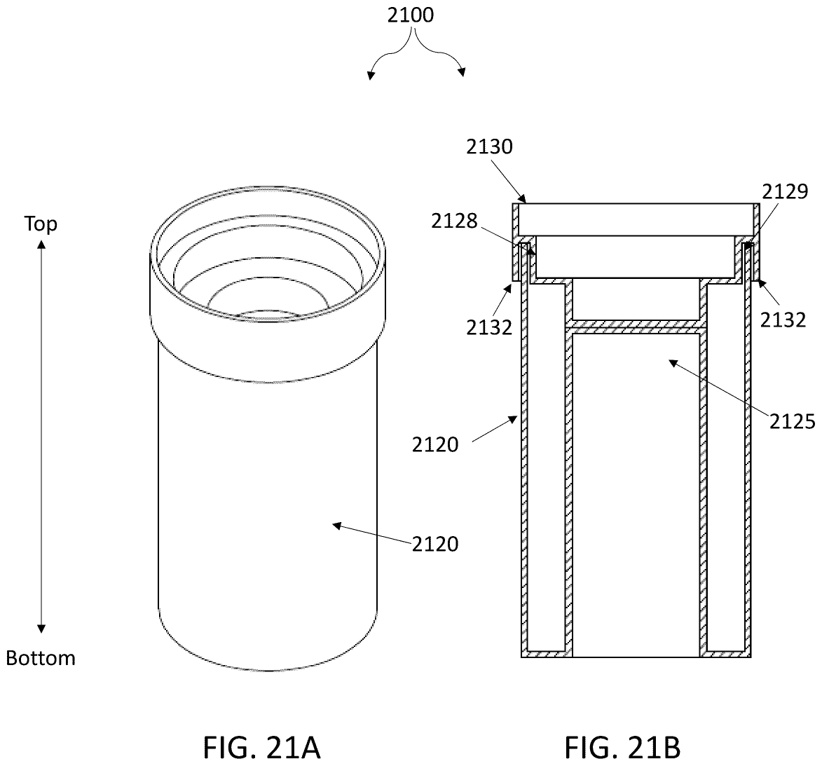

Moreover, as noted above, in some variations, the deactivation container body may have an annular shape. For example, as shown in FIG. 21A, an annular container 2100a may have a body 2120 that is cylindrical or substantially cylindrical. Referring to FIG. 21B, the body 2120 and a lid 2130 of the annular container 2100b may be configured to releasably couple via one or more coupling mechanisms, such as one or more of a mechanical coupling (e.g., via threads, snap fit) and a friction fit (e.g., interference fit, clearance fit, transition fit). In some variations, the body 2120 and the lid 2130 of the annular container 2100b may include engagement features (e.g., threads) for engaging complementary features (e.g., threads) of the lid 2130 for releasably coupling the body 2120 and the lid 2130. The engagement feature may be on an external or internal surface of the annular container body 2120. In some variations, a cavity 2125 of the annular body 2120 may be configured to receive at least a portion of the lid 2130 therein.

In general, an annular deactivation container may not require a support for maintaining a predetermined (e.g., upright) position of the container. For example, a bottom of the annular container may be a planar surface with a diameter or major axis that enables the annular container to balance on another surface (e.g., another planar surface). In some variations, the diameter or major axis of the annular container may be about 1 inch to about 10 inches (including all values and sub-ranges therein). For example, the diameter or major axis of the annular container may be about 1 inch to about 2 inches, about 1 inch to about 3 inches, about 1 inch to about 4 inches, about 1 inch to about 5 inches, about 1 inch to about 6 inches, about 1 inch to about 8 inches, or about 1 inch to about 9 inches. The diameter or major axis and/or minor axis of the annular container may vary along a longitudinal axis of the container body or may remain constant.

### b. Alignment Feature

The deactivation container body may further comprise one or more alignment features configured to interact with a device received by the deactivation container body to appropriately position and/or hold the device within the deactivation container body for a safe and effective deactivation process. The deactivation container body may comprise a single alignment feature, or a plurality of alignment features, such as 2, 3, 4, 5, 6, 7, 8, 9, 10 or more alignment features. The device described herein (e.g., the alkali metal device) may be placed or inserted into the cavity of the container body such that the device contacts the alignment feature rather than the sidewall(s) and/or bottom that define the cavity. The alignment feature may be configured to maintain a gap between the device and the sidewalls and/or bottom of the cavity, which may advantageously allow for fluid to flow around substantially all of the alkali metal device. In this way, a fluid such as an alkali metal reactant or solubilizer may react with substantially all of the remaining alkali metal of the alkali metal device.

The alignment feature may be integrally formed with, or coupled to, an inner surface of the deactivation container body (i.e., within the deactivation container cavity). The alignment feature may comprise one or more of a protrusion, rod, ridge, bump, and the like. The alignment feature may extend, continuously or discontinuously, vertically (i.e., along a longitudinal dimension of the deactivation container body) or horizontally (i.e., along a lateral dimension of the deactivation container body). For example, the alignment feature may comprise a protrusion integrally formed with a sidewall such that it extends from a surface of the sidewall into the cavity. The alignment feature may extend longitudinally from the bottom of the cavity to, near, or beyond the top of the cavity. In other variations, the alignment feature may extend along only a portion of the longitudinal dimension of the cavity. For example, the alignment feature may start at a location approximately one-quarter, one-third, or one-half along the longitudinal dimension of the cavity. In a variation with a plurality of alignment features, there may be multiple alignment features aligned colinearly, either laterally or longitudinally, that are spaced apart from one another. The colinear arrangement may extend along the longitudinal dimension and/or lateral dimension of the cavity. For example, the alignment feature may extend horizontally (i.e., along the lateral dimension) from a first sidewall to a second sidewall of the cavity. In other variations, the alignment feature may extend along only a portion of the lateral dimension of the cavity. For example, the alignment feature may start at a location approximately one-quarter, one-third, or one-half along the lateral dimension of the cavity. In yet another variation, the alignment feature may extend from the bottom of the deactivation container body. In a variation with alignment features on the sidewalls, the alignment features may be spaced laterally (horizontally) based on a width of the patch (i.e., by a distance that is less than the width of the patch), vertically based on the height of the patch (i.e., by a distance that is less than the height of the patch), or on the bottom of the container, the alignment features may be dimensioned to be longer than a thickness of the patch, or may be positioned relative to one another based on a length of the patch (i.e., a distance between the alignment features may be less than the length of the patch). When a series of alignment features are present (as in a set), the previous descriptions may refer to the first and last alignment features of the set.

In this way, the patch may be maintained in a position within the cavity that may advantageously provide a gap between the device and a sidewall of the deactivation container body. The gap may allow for uniform exposure of an alkali metal reactant or solubilizer to the alkali metal device, such that the resulting reaction is uniform across an alkali metal layer of the alkali metal device. The gap may also allow the alkali metal reactant or alkali metal solubilizer to circulate within the deactivation container and around the alkali metal device, which may result in deactivating substantially all of the previously unreacted alkali metal thereof and/or transfer heat from the alkali metal into the alkali metal reactant or alkali metal solubilizer. Transferring heat from the alkali metal into the alkali metal reactant or solubilizer may minimize the temperature increase of the alkali metal resulting from the reaction therebetween. A rate of the reaction between the alkali metal and the alkali metal reactant or solubilizer may increase as the temperature of the alkali metal increases, so minimizing the increase of alkali metal temperature may advantageously keep the alkali metal below its melting point (e.g., in a variation in which the alkali metal comprises sodium, may be about 100 degrees C.). The gap may also be configured to inhibit, decrease, or otherwise minimize heat transfer between an exothermic reaction occurring on or near a surface of the alkali metal device and one or more sidewalls of the body. For example, each sidewall of the deactivation container body may be maintained below about 50 degrees C. In other variations, each sidewall may be maintained below about 30 degrees C., about 40 degrees C., about 45 degrees C., about 55 degrees C., or about 60 degrees C. This may advantageously allow a user to handle the deactivation container during and/or after the reaction.

Referring back to FIG. 3, an exemplary variation of an alignment feature within a cavity of a container body 220 is shown. As shown, the body may comprise a first alignment feature 260 and a second alignment feature 260. The first and second alignment features 260 may be configured to contact a device such as, for example, an alkali metal device and may be positioned relative to one another based on a configuration (e.g., dimension, shape) of the alkali metal device. For example, as shown in FIG. 3, the first and second alignment features 260 may be separated from one another based on a width of the alkali metal device, such that at least two portions (e.g., edges) of the alkali metal device are supported by or otherwise contact the alignment features 260. In this way, the alignment features 260 may maintain a position of the alkali metal device within the cavity of the body 220 or may otherwise limit the range of motion of the alkali metal device such that a gap between the alkali metal device and one or more sidewalls of the body 220 is maintained. The gap may be configured to inhibit, decrease, or otherwise minimize heat transfer between an exothermic reaction occurring on or near a surface of the alkali metal device and one or more sidewalls of the body 220.

### c. Lid

The deactivation container may comprise a lid configured to cover the opening of the body of the deactivation container. The lid may be configured to engage with the body, and/or with the support coupled thereto, such that a fluidic seal is formed therebetween. The lid may be configured to withstand a force (e.g., a pressure from within or an externally applied force) without buckling, failing, deforming, degrading, and/or corroding. The lid may comprise one or more pressure management features, such as a vent and/or a valve (e.g., a pressure relief valve), configured to release pressure from within the deactivation container to an external environment. The pressure management feature may comprise a one-way valve or a bi-directional valve. In some variations, the pressure management feature may comprise, for example, a vent configured to enable fluid exchange between a cavity of the deactivation container and an external environment thereby releasing pressure from within the deactivation container, and a membrane configured to cover the vent. The membrane may be configured to limit fluid (e.g., liquid and/or gas) exchange between the cavity of the deactivation container and the external environment through the vent. As another example, the pressure management feature may comprise a one-way valve, such as an umbrella valve. As such, the lid may allow fluid to flow from within the body to an external environment. The fluid flowing through the pressure management feature may be a gaseous byproduct of an exothermic reaction between an alkali metal device and an alkali metal reactant contained within the body. The fluid may flow through the pressure management feature when the lid is engaged with the body (as in the closed position). Separately, in some variations, the pressure management feature may comprise a closed position, where no fluid may flow therethrough even when the lid is the closed position. In some variations, the pressure management feature may be maintained in the closed position by a security mechanism releasably coupled to one or more of the pressure management feature and the lid.

The lid may be made of a material capable of withstanding the forces and/or chemical reaction byproducts described herein, such as a plastic (e.g., polypropylene, polycarbonate, polystyrene, polyethylene), a metal (e.g., stainless steel, steel, aluminum, copper), a glass, a rubber (e.g., nitrile, neoprene, ethylene propylene, silicone, fluorocarbon), or combination thereof. Moreover, the lid may be shaped and sized to fit at least partially within and/or at least partially over the body. For example, for a deactivation container having a substantially rectangular or square body and opening thereof, the lid may have a substantially rectangular or square shape such that it may cover the opening. As another example, for a deactivation container having a substantially round or circular (e.g., annular) body and opening thereof, the lid may have a substantially round or circular shape such that it may cover the opening.

In some variations, the lid may include one or more features to enhance the ability of the lid to withstand forces and/or chemical reaction byproducts described herein. For example, the lid may have a variable cross-sectional thickness, where a thickness of the lid is greatest at a central point or portion thereof and smallest at its ends, to help reduce pressure on a bottom of the lid due to chemical reaction byproducts described herein. Additionally, or alternatively, the lid may include one or more features to increase a stiffness of the lid, such as one or more ridges or ribs along a top of the lid, to bolster the ability of the lid to withstand pressure buildup within the body cavity.

In general, the lid may be rotatably and/or releasably coupled to a deactivation container body, and/or to a support coupled to the body, such that the lid may be transitioned between a closed position and an open position. As shown and described with reference to FIG. 19 and FIG. 20 herein, in some variations, the lid may couple directly to the support. When the support is integrally formed with the deactivation container body, a lid coupled directly to a support may also be coupled to the body. Similarly, in such variations, a lid coupled directly to the body may also be coupled to the support. When the support is not integrally formed with the body, the lid may be coupled to one or both of the support and the body. For example, the lid may be coupled to one of the support or the body when the support and the body are separate components. As another example, the lid may be coupled (e.g., via one or more separate coupling mechanisms) to each of the support and the body when the support and the body are separate components.

The lid may be opened to and maintained at a predetermined angle relative to the body (e.g., a plane defining the opening of the body, such as a transverse plane of the body) via a stop coupled to one or more of the lid, the body, and the support. In some variations, the predetermined angle may be about 90 degrees to about 270 degrees, about 90 degrees to about 180 degrees, about 90 degrees to about 170 degrees, about 90 degrees to about 160 degrees, about 90 degrees to about 150 degrees, about 90 degrees to about 140 degrees, or about 90 degrees to about 135 degrees. In the closed position, the longitudinal axis of the lid may be positioned parallel or substantially parallel to a longitudinal axis of the opening of the body.

In one example, the support may be a bridge, and the lid may be rotatably and/or releasably coupled to the bridge via one or more coupling mechanisms. Such an example is shown in FIG. 20, where the lid 2030 is rotatably coupled to a bridge 2010 that extends from a body 2020 of the deactivation container 2015. The lid 2030 may be configured to move from the closed position (depicted) to an open position to allow access to a cavity (not shown) of the body 2020 while allowing for the cavity to be covered as desired. The lid 2030 may move via a hinge 2024 that defines a pivot point about which the lid 2030 may rotate. The hinge 2024 may be formed with a rod 2033 of the bridge 2010, and a corresponding hinge clip 2050 of the lid 2030. The hinge clip 2050 may be coupled to (e.g., integrally formed with) a first end 2001 of the lid 2030. The rod 2033 and hinge clip 2050 may engage with one another to form the hinge 2024 to couple (e.g., releasably and rotatably) the lid 2030 to the bridge 2010. For example, the hinge clip 2050 may releasably receive the rod 2033 and may pivot thereabout. The hinge clip 2050 may releasably engage the rod 2033 in any suitable manner, such as, for example via a friction force, a compressive force, or a combination thereof. In some variations, the hinge clip 2050 may fully circumferentially surround the rod such that the lid 230 is rotatably coupled to the body 2020 but may not be releasable therefrom. It should be appreciated that while the first and second coupling features are described above as a rod and a clip respectively, the first and second coupling features may be or may include any suitable elements that allow for relative motion between the lid 2030 and the bridge 2010. Moreover, in some variations, the coupling feature of the lid may comprise a rod, and the coupling feature of the body may comprise a clip. Additionally, it should be understood that, while the rod and hinge clip may be described as coupled to the bridge, one or both of the rod and hinge clip may additionally or alternatively be positioned on the container body.

As explained above, the lid 2030 may be configured to transition between a closed position and an open position via the hinge 2024. In the closed position, the lid 2030 may be engaged with the bridge 2010 along a perimeter of the lid 2030 and a perimeter of the bridge 2010 (e.g., a frame comprising the bridge 2010), while in the open position, the perimeter of the lid 2030 and the perimeter of the bridge 2010 may not be engaged and may instead be spaced apart. The lid 2030 may comprise additional components associated with releasing the lid from the closed position. For example, in some variations, the lid 2030 may comprise a lock configured to maintain the lid 2030 in closed configuration. The lock may include one or more of a latch, hook, a clasp, a button, a spring, and a clip. In some variations, the lock may comprise a latch 2032. The latch 2032 may be positioned at a second end 2003 of the lid 2030 opposite a first end 2001. In some variations, the latch 2032 may be positioned at any location along the perimeter of the lid 2030. The latch 2032 may be a protrusion in one or more directions that extends from an external surface of the lid 2030. In some variations, the latch 2032 may be deformable (e.g., elastic) such that it may be released from an engagement feature (e.g., a sidewall) of the bridge 2010. For example, in some variations, a corresponding engagement features of the bridge 2010 may be configured to releasably engage the latch 2032. The corresponding feature may be, for example, a sidewall, an opening, an indent, a groove, a depression, a clip, a hook, or any other suitable feature of the bridge 2010. In some variations, as will be discussed in detail below, the latch 2032 may releasably engage a sidewall of the bridge 2010 such that the lid 2030 is retained in a closed position even if the body 2020 is, for example, turned upside down.

Additionally, or alternatively, the lid may be coupled to the body of the deactivation container. Referring again to FIG. 2, the body 220 may be rotatably and/or releasably coupled to a lid 230, shown in a closed position. The lid 230 may be configured to move from the closed position to an open position to allow access to the cavity of the body 220 while allowing for the cavity to be covered as desired. In some variations, the lid 230 may be rotatably coupled to the body 220. For example, the lid 230 may move via a hinge, such as a hinge 224, which may be positioned at any suitable location on the deactivation container, such as, for example, adjacent to an opening thereof. The hinge 224 may define a pivot point about which the lid 230 may rotate. The hinge 224 may be formed from a first coupling feature, such as, for example, a rod 225 of the body 220, and a second, corresponding coupling feature, such as a hinge clip 250, of the lid 230. The first and second coupling features may engage with one another to form the hinge 224 thereby coupling, here releasably and rotatably, the lid 230 to the body 220. For example, the hinge clip 250 may releasably receive the rod 225 and may pivot thereabout. The hinge clip 250 may releasably engage the rod 225 in any suitable manner, such as, for example via a friction force, a compressive force, or a combination thereof. In some variations, the hinge clip 250 may fully circumferentially surround the rod such that the lid 230 is rotatably coupled to the body 220, but may not be releasable therefrom. It should be appreciated that while the first and second coupling features are described above as a rod and a clip respectively, the first and second coupling features may be or may include any suitable elements that allow for relative motion between the lid 230 and the body 220. Moreover, in some variations, the second coupling feature (the coupling feature of the lid) may comprise a rod, and the first coupling feature (the coupling feature of the body) may comprise a clip.

The lid 230 may be configured to transition between a closed position and an open position via the hinge 224. In the closed position, the lid 230 may be engaged with the body 220 along a perimeter of the lid 230 and a perimeter of the body 220, while in the open position, the perimeter of the lid 230 and the perimeter of the body 220 may not be engaged and may instead be spaced apart. The lid 230 may comprise additional components associated with releasing the lid from the closed position. For example, in some variations, the lid 230 may comprise a lock configured to maintain the lid 230 in closed configuration. The lock may include a hook, a clasp, a button, a spring, or a combination thereof. In some variations, the lock may comprise a latch 232. The latch 232 may be positioned at a second end 270B of the lid 230, where the second end is opposite a first end 270A. In some variations, the latch may be positioned at any location along the perimeter the lid 230. The latch 232 may be a protrusion in one or more directions that extends from an external surface of the lid 230. In some variations, the latch 232 may be deformable (e.g., elastic) such that a user may release the latch 232 from a sidewall of the body 220. For example, in some variations, there may be a corresponding feature of the sidewall of the body 220 that is configured to receive the latch 232. The corresponding feature may be, for example, an indent, a groove, a depression, a clip, a hook, or any other suitable feature. The latch 232 may releasably engage a sidewall of the body 220 such that the lid 230 is retained in a closed position even if the body 220 is, for example, turned upside down.

The lid of the deactivation container may transition from a closed position to an open position. FIG. 3 shows an exemplary variation of a deactivation container 215 with a lid 230 in an open position. The lid 230 may be opened to and maintained at a predetermined angle relative to the body 220 (e.g., the plane defining the opening of the body) via a stop coupled to one or more of the lid 230 (e.g., clip 250) and the body 220 (e.g., rod 225). In some variations, the predetermined angle may be about 90 degrees to about 270 degrees, about 90 degrees to about 180 degrees, about 90 degrees to about 170 degrees, about 90 degrees to about 160 degrees, about 90 degrees to about 150 degrees, about 90 degrees to about 140 degrees, or about 90 degrees to about 135 degrees.

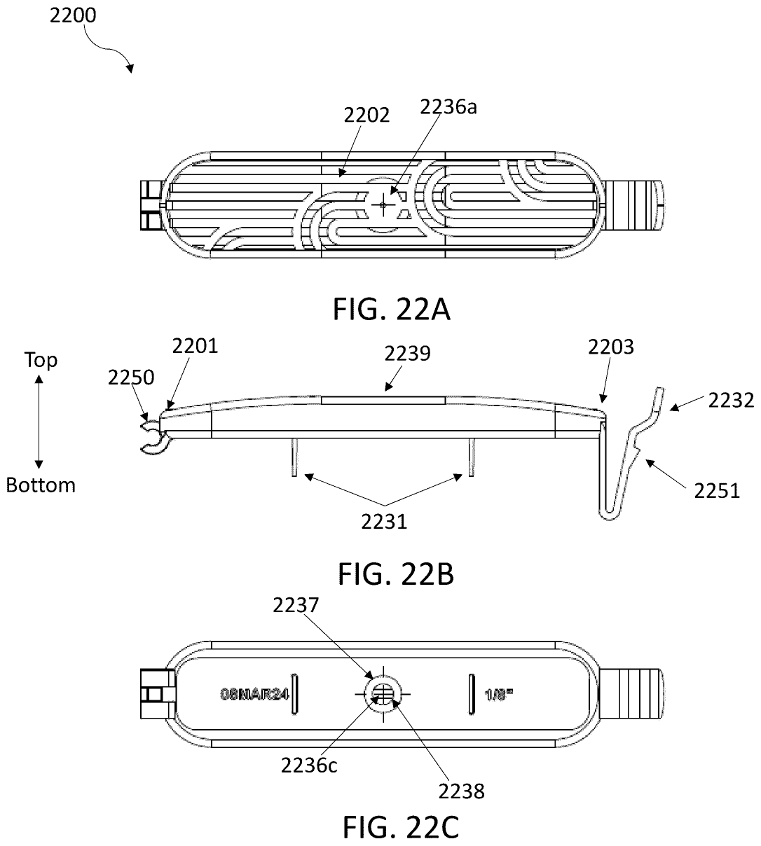

FIGS. 22A-22C illustrate an exemplary variation of a lid 2200. Referring to FIG. 22A, which shows a top view of the lid 2200, the lid 2200 may comprise one or more structural features, such a ridge 2202, which may extend longitudinally along the lid 2200 to increase the structural rigidity of the lid 2200. That is, the ridge 2202 may reduce and/or resist shear stress as the lid 2200 responds to pressure build-up within the cavity of the deactivation container, thus allowing the lid 2200 to withstand a greater amount of pressure within the cavity. In some variations, the lid 2200 may comprise a plurality of ridges 2202 (e.g., two, three, four, or more) forming channels (e.g., indents, depressions, or grooves) therebetween. One or more (including all) of the plurality of ridges 2202 may be parallel to one another. The ridge 2202 may comprise a length, a width, and a depth, each of which may be less than a length, a width, and a depth of the lid 2200. Additionally, or alternatively, the lid 2200 may be shaped to optimize pressure distribution along the lid 2200. For example, as shown in FIG. 22B, a thickness of the lid 2200 may vary along a longitudinal axis thereof to advantageously distribute pressure along the bottom of the lid 2200. In particular, thickness of the lid 2200 may be smallest at first and second ends 2201,2203 of the lid 2200 and may increase longitudinally toward a central portion 2237 thereof, where the thickness may be greatest.

Also shown in FIG. 22B are engagement features extending therefrom, such as a hinge clip 2250 and a latch 2232. The hinge clip 2250 may be positioned at a first end 2201 of the lid 2200 and the latch 2232 may be positioned at a second (e.g., opposite) end 2203 of the lid 2250, where the second end is opposite the first end. The hinge clip 2250 may be configured to releasably engage a hinge of the container body via a friction force, a compressive force, or a combination thereof. In some variations, the hinge clip 2250 may comprise a C-shape. The lid 2200 may be configured to transition between a closed position and an open position via the hinge formed with the hinge clip 2250.

In the closed position, the latch 2232 may be configured to releasably engage a portion of the support (e.g., a bridge). The latch 2232 may comprise a protrusion with a U-shaped bend formed between a movable arm 2252 and a fixed arm 2253 of the latch 2232. The U-shaped bend may allow for an elastic force to cause the latch 2232 to securely engage the lid 2200 to the support (e.g., bridge). That is, the latch 2232 may have an at-rest configuration in which the latch 2232 naturally engages the support, and a flexed configuration in which the latch 2232 is disengaged from the support. In particular, as will be described in more detail herein, the latch 2232 may include a “stop” element, ledge 2251, configured to abut a bottom sidewall of the support (not shown) in the at-rest configuration. Thus, the engagement of the ledge 2251 and bottom sidewall of the support may prevent the latch 2232, and thus the lid 2230, from deviating from the intended closed position. To subsequently move the lid 2200 to the open position, a movable arm 2252 of the latch 2232 may be moved toward a fixed arm 2253 of the latch, thereby disengaging the ledge 2251 from the bottom sidewall of the support and allowing adjustment of the lid 2200 with respect to the container body.

FIGS. 22A and 22C illustrate an exemplary pressure management feature for use with a lid of the deactivation containers described herein, such as, for example the lid 2200. Here, the pressure management feature may include a vent that extends transversely through the lid 2200. The vent may have a first opening 2236a at a top of the lid 2200, a lumen (not shown) extending through the lid 2200, and a second opening 2236b at a bottom of the lid. The vent may be configured to provide a path for fluid flow out of the cavity and into an external environment. The pressure management feature may further include a membrane 2238 configured to cover the second opening 2236. The membrane 2238 may be configured to control (e.g., limit) fluid exchange between the container body cavity and the external environment. In some variations, the membrane 2238 may have a pore size that allows a gaseous byproduct to permeate therethrough but does not allow the liquid within the cavity to permeate therethrough. For example, in some variations, the pore size may be about 0.00001 μm to about 50 μm, such as about 0.0001 μm to about 45 μm, about 0.001 μm to about 40 μm, about 0.01 μm to about 35 μm, about 0.1 μm to about 30 μm, about 1 μm to about 25 μm, about 5 μm to about 20 μm, or about 10 μm to about 15 μm (including all ranges and subranges in between). The membrane 2238 may have a pressure rating (e.g., a predetermined pressure rating) that allows the gaseous byproduct to permeate therethrough before the pressure buildup within the cavity causes damage to the deactivation container. The pressure rating may be about 1 psi to about 16 psi, such as about 2 psi to about 14 psi, about 3 psi to about 12 psi, about 4 psi to about 10 psi, or about 5 psi to about 8 psi (including all ranges and subranges therebetween).

Further, referring again to FIG. 22B, the lid 2200 may include one or more projections 2231 (e.g., two, three, four, five, or more than five projections) extending transversely from a bottom of the lid 2200. The projections 2231 may be spaced apart and positioned at different longitudinal locations along the bottom of the lid 2200. The projections 2231 may increase a resistance of the lid 2200 against shear stress (e.g., torsional stress), which may occur due to pressure buildup within the cavity. Additionally, or alternatively, the projections 2231 may be configured to traverse an opening of a deactivation container and extend into the cavity of the deactivation container. In some cases, a device within the cavity may have a density that is less than a density of the fluid therein. Accordingly, in some variations, the projections 2231 may extend at least partially into the volume of fluid within the cavity to maintain submersion of the device within the fluid inside the container. Put another way, in the closed position, the projections 2231 may be configured to extend to or at least partially into the fluid such that the projections 2231 may prevent the device from emerging from the fluid. That is, the projections 2231 may be configured to contact the device within the deactivation container when the lid is in the closed position. In some variations, the projections 2231 may also or alternatively be configured to occupy a predetermined volume of the cavity, such as, for example, a predetermined volume of at least about 75% of the volume of the cavity. In other variations, the predetermined volume may be about 10%, about 20%, about 30%, about 40%, about 50%, about 60%, or about 70%. The projections 2231 may have a length of about equal to or less than 2.5 cm, such as a length about equal to or less than 2.25 cm, about equal to or less than 2 cm, about equal to or less than 1.75 cm, about equal to or less than 1.5 cm, about equal to or less than 1.25 cm, about equal to or less than 1 cm, about equal to or less than 0.75 cm, or about equal to or less than 0.5 cm.

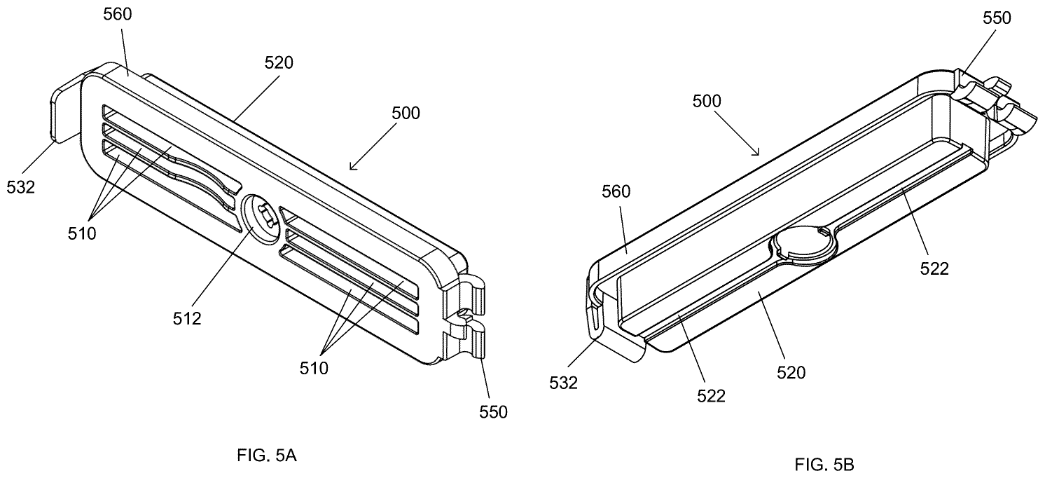

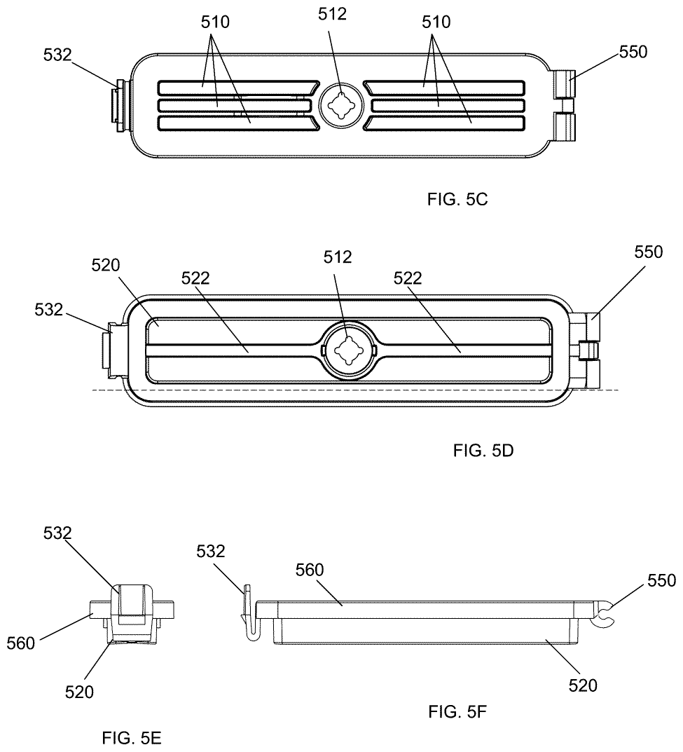

FIGS. 5A-F illustrate another exemplary variation of a lid 500. The lid 500 may comprise one or more structural features on a top side of the lid 500. For example, the lid 500 may comprise a channel 510, which may be configured to increase the structural rigidity of the lid. The channel 510 may extend parallel to a longitudinal axis of the lid 500. In some variations, the lid may comprise a plurality of channels (e.g., two, three, four, or more). Each of the plurality of channels 510 may be parallel to one another. The channel 510 may form an indent, depression, or groove on a top surface of the lid 500. The channel 510 may comprise a length, a width, and a depth, each of which may be less than a length, a width, and a depth of the lid 500. In this way, the lid 500 may be able to withstand a greater amount of pressure within the cavity because the channel 510 may reduce and/or resist shear stress as the lid 500 bends in one or more directions in response to pressure build-up within the deactivation container body.

The lid 500 may further comprise one or more fluid control features on a bottom side of the lid 500. The fluid control features may be configured to reduce cavity volume and/or direct fluid (e.g., in gaseous state) towards another component of the lid 500. For example, the lid 500 may comprise a portion 520, which may comprise a projection extending along a perimeter of the lid 500 that is configured to reduce the volume of the cavity when the lid is in its closed position. A reduced cavity volume may advantageously minimize a gap between a liquid contained within the cavity and the lid 500, which may subsequently reduce the maximum volume of gaseous byproduct of an exothermic reaction that may be ultimately stored within the container following release via, for example, a pressure relief valve in the lid 500. Accordingly, the portion 520 may be configured to traverse an opening of a deactivation container (not shown) and may occupy a predetermined volume of a cavity of the deactivation container. In an exemplary variations, the predetermined volume may be at least 75% of the volume of the cavity. In some variations, the predetermined volume may be about 1% to about 80%, such as about 10%, about 20%, about 30%, about 40%, about 50%, about 60%, about 70%, or about 80% of the total cavity volume.

When the liquid (e.g., the alkali metal reactant or solubilizer described herein) is present in the deactivation container, the deactivation container may have a head space, which refers to the volume of the container that is not filled with liquid. Put differently, the head space refers the volume between the top surface of the liquid and a plane at the top of the container body (e.g., a plane intersecting the top edges of the sidewalls). In some variations, when the lid is in the closed position, part of the head space may be occupied by the portion 520. For example, in some variations, the portion 520 may occupy about 50% to about 99% or about 100% of the head space, about 60% to about 95%, or about 75% to about 95%. In an exemplary variation, the portion 520 may occupy about 90% of the head space.

The portion 520 may comprise a fluid channel 522 configured to direct fluid flow towards one or more components of the lid 500. In some variations, the portion 520 may comprise a plurality of fluid channels. The fluid channel 522 may comprise a groove, a depression, or an indent through or along a surface of the portion 520. The fluid channel 522 may or may not be centered relative to a width of the lid 500. The fluid channel 522 may extend along the entirety of a longitudinal dimension of the portion 520. In some variations, there may be one or more fluid channels 522 extending along a lateral dimension of the portion 520. In further variations, there may be a combination of fluid channels 522 extending along the longitudinal dimension and the lateral dimension of the portion 520. In yet another variation, a segment of a fluid channel 522 may extend along the lid's longitudinal dimension and another segment of the fluid channel 522 may extend along the lid's lateral dimension. The fluid channel 522 may be configured to direct fluid towards an opening 512 that is configured to receive a valve (e.g., the pressure relief valve). The opening 512 may, in some variations, interpose one or more channels 510 and/or fluid channel 522 of the lid 500. The opening may extend from the top side of the lid 500 to the bottom side of the lid 500. The opening may be positioned at a midpoint of the longitudinal dimension of the lid 500. In some variations, the opening may be positioned at a midpoint of the lateral dimension of the lid 500. For example, the fluid channel 522 may direct a byproduct of an exothermic reaction between an alkali metal and an alkali metal reactant or solubilizer towards a pressure relief valve received within the opening 512.

Similar to the description above for lid 230, the lid 500 may include one or more engagement features extending from a side of the lid, such as a hinge clip 550 and a latch 532. The hinge clip 550 may be positioned at a first end of the lid 500 and the latch 532 may be positioned at a second end of the lid 500, where the second end is opposite the first end. The hinge clip 550 may be configured to releasably engage a hinge of the container body via a friction force, a compressive force, or a combination thereof. In some variations, the hinge clip 550 may comprise a C-shape. The lid 500 may be configured to transition between a closed position and an open position via the hinge and hinge clip 550. When in the closed position, the latch 532 may be configured to releasably engage a sidewall of the body. The latch 532 may comprise a protrusion with a U-shaped bend such that an elastic force may be applied to the sidewall of the deactivation container body and thus securely engage the lid 500 to the body. There may be a corresponding feature of the sidewall of the body that is configured to receive the latch 532, such as a latch receiver. In some variations, the corresponding feature may be, for example, an indent, a groove, a depression, a clip, or a hook.

The lid 500 may comprise a length, a width, and a depth that may correspond to an opening of the deactivation container described herein. In some variations, the length may be about 1 inch to about 6 inches, about 2 inches to about 6 inches, about 3 inches to about 6 inches, about 4 inches to about 6 inches, or about 4 inches to about 5 inches. In some variations, the width may be about 0.5 inches to about 1.5 inches, about 0.5 inches to about 2 inches, about 0.5 inches to about 3 inches, about 0.5 inches to about 4 inches, about 0.5 inches to about 5 inches, or about 0.5 inches to about 6 inches. In an exemplary variation, the width may be about 1 inch. In some variations, the depth may be about 0.25 inches to about 0.75 inches, 0.25 inches to about 1 inch, 0.25 inches to about 1.5 inches, or 0.25 inches to about 2 inches.

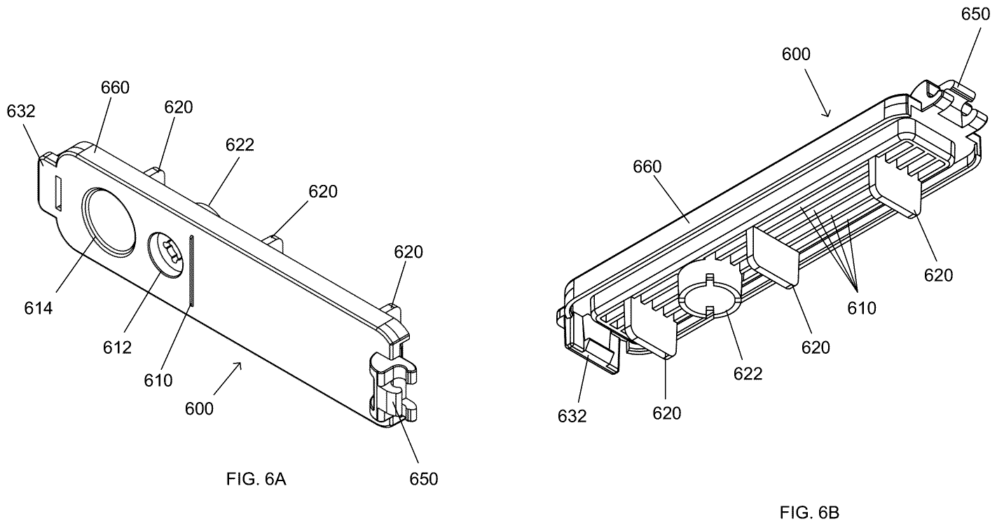

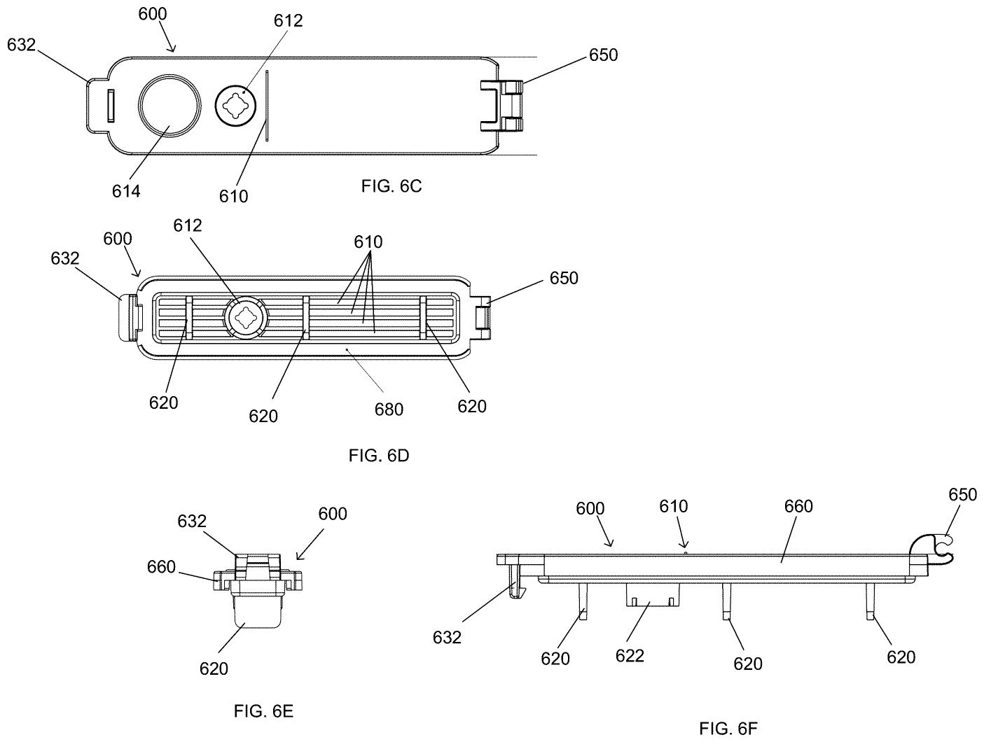

FIGS. 6A-F illustrates an alternative variation of a lid 600 with projections extending across a width of the lid 600. The projections may be configured to increase the structural rigidity of the lid 600 and/or reduce the cavity volume. For example, the lid 600 may comprise a portion 620 comprising a projection or, some variations, a plurality of projections. Each of the portions 620 may be positioned at a different location along a longitudinal dimension of the lid 600. The portion 620 may increase ability of the lid 600 to resist shear stress (e.g., torsional stress), which may be applied by the pressure within the cavity. Additionally, like the portion 520 described above, the portion 620 may be configured to traverse an opening of a deactivation container (not shown) and may occupy a predetermined volume of a cavity of the deactivation container (which may or may not include the predetermined volume occupied by the valve receiver protrusion 622). In some cases, a device within the cavity may have a density that is less than a density of the fluid therein. Accordingly, in some variations, the portion 620 may extend at least partially into the cavity to hold the device submerged within the fluid. In some variations, the predetermined volume may be at least about 75% of the volume of the cavity. In other variations, the predetermined volume may be about 10%, about 20%, about 30%, about 40%, about 50%, about 60%, or about 70%. The lid 600 may further define a fluid channel 610 configured to direct fluid flow towards a pressure relief valve in the lid 600 (not shown). For example, the fluid channel 622 may direct a byproduct of an exothermic reaction between an alkali metal and an alkali metal reactant towards a pressure relief valve received within a valve receiver protrusion 622. The fluid channel 610 may comprise a groove, a depression, or an indent through or along a surface of the bottom side of the lid 600. The fluid channel 610 may extend parallel to a longitudinal dimension of the lid 600.

The lid 600 may comprise one or more pressure management features on a top side of the lid 600. For example, the lid 600 may further comprise a valve receiver 612 configured to receive a valve, such as a pressure relief valve. The valve receiver 612 may comprise an opening within which the pressure relief valve may be inserted and/or coupled. The opening may extend from the top side of the lid 600 to the bottom side of the lid 600. As described above, on a bottom side of the lid 600 may be the valve receiver protrusion 622. In this way, the opening may extend from the valve receiver 612 to the valve receiver protrusion 622. Each of the valve receiver 612 and valve receiver protrusion 622 may be positioned at a location along the longitudinal dimension of the lid 600. In other variations, the valve receiver 612 and valve receiver protrusion 622 may be positioned at any point along the longitudinal dimension of the lid 600. The lid 600 may further comprise an indent 614 configured to receive a user's finger such that the user may apply a force under a portion of a security mechanism (not shown) that may cover the pressure relief valve. The force applied may be sufficient to remove at least a portion of the security mechanism. In this way, the security mechanism may be removed so that the pressure relief valve within the valve receiver 611 may transition to an open position based upon a predetermined pressure differential between the cavity pressure and the external environment pressure. Corresponding to the security mechanism may be a protrusion 610, which may be configured to receive or abut a security mechanism configured to cover a portion of a pressure relief valve. In this way, the security mechanism may not slide or otherwise move along the longitudinal length of the lid 600 once it is positioned over the pressure relief valve. The protrusion 610 may partially or completely extend across a width of the lid 600.

Like the lid 2200 of FIGS. 22A-22C and the lid 500 depicted in FIGS. 5A-5F, the lid 600 may include one or more engagement features extending from a side of the lid, such as a hinge clip 650 and a latch 632. The hinge clip 650 and latch 632 may operate and may be positioned as previously described. The latch 632 may have an alternative configuration than the variation 532. For example, the latch 632 may comprise a protrusion with an edge that is configured to releasably engage a sidewall of the body. For example, there may be a corresponding feature of the sidewall of the body that is configured to receive the latch 632, such as a latch receiver. The corresponding feature may be, for example, an indent, a groove, a depression, a clip, a hook, or any other suitable feature. In this way, a user may apply a force to the latch 632 to tilt the sharp edge away from the corresponding feature of the body, and thus disengage the latch 632 from the body.

The lid 600 may comprise a length, a width, and a depth that may correspond to an opening of the deactivation container described herein. In some variations, the length may be about 1 inch to about 2 inches, about 1 inch to about 3 inches, about 1 inch to about 4 inches, about 1 inch to about 5 inches, or about 1 inch to about 6 inches. In an exemplary variation, the length may be about 4.37 inches. In some variations, the width may be about 0.5 inches to about 1 inch, about 0.5 inches to about 2 inches, about 0.5 inches to about 3 inches, about 0.5 inches to about 4 inches, about 0.5 inches to about 5 inches, or about 0.5 inches to about 6 inches. In an exemplary variation, the width may be about 0.98 inches. In some variations, the depth may be about 0.25 inches to about 1 inch, about 0.25 inches to about 1.5 inches, or about 0.25 inches to about 2 inches. In an exemplary variation, the depth may be about 0.81 inches.

In some variations, an engagement feature of the lid may include threads. For example, turning back to FIG. 21B, in some variations, the lid 2130 and the deactivation container body 2120 may both include annular shapes. Accordingly, the lid 2130 may include first threads (not shown) configured to engage complementary second threads (not shown) of the body 2120. The first threads may encircle (or at least partially encircle) a surface of the lid 2130, such as an annular sidewall 2128 extending distally from the lid 2130. Oppositely, the second threads may encircle (or at least partially encircle) a surface of the body 2120, such as a proximal portion of an annular sidewall 2129 of the body 2120. In some variations, the annular sidewall 2128 may be an exterior sidewall of the lid 2130 and first threads may be external threads, while the annular sidewall 2129 may be an interior sidewall of the body 2120 and second threads may be internal threads. Accordingly, to transition the lid 2130 from the closed position (depicted in FIG. 21B) to an open position, the lid 2130 may be rotated and extracted from and/or off of the annular body 2120.

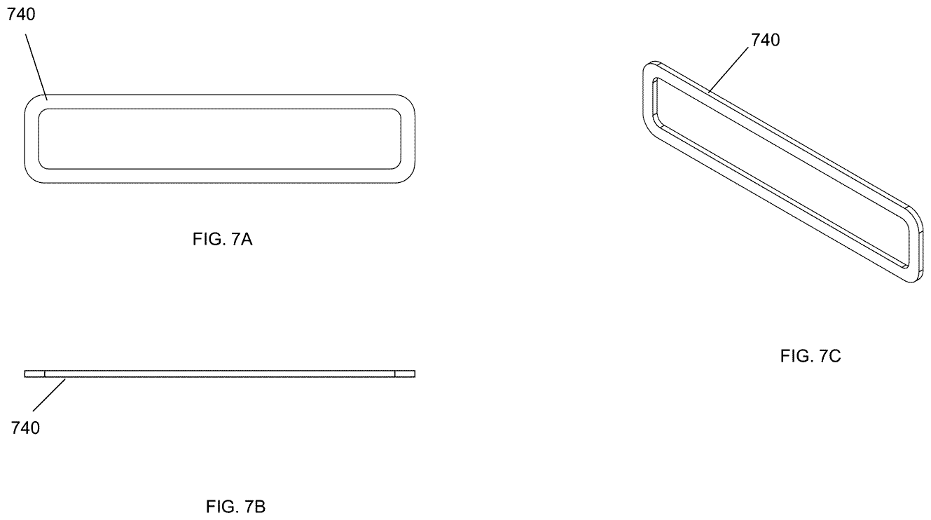

In some variations, the deactivation container described herein may comprise one or more fluidic sealing components. For example, a lid of the deactivation container may comprise a gasket 740 as shown in FIGS. 7A-C. The gasket 740 may extend along a perimeter of a bottom side of the lid. In this way, the gasket may be adjacent to a portion of the lid such that the gasket engages an edge of at least one sidewall of the container body when the lid is in the closed position. The gasket depicted in FIGS. 7A-7C may be configured to engage with at least the embodiments of the lid shown in FIGS. 5A-F or FIGS. 6A-F. The gasket 740 may be a deformable material configured to create a fluidic seal, such as rubber (e.g., nitrile, neoprene, ethylene propylene, silicone, fluorocarbon), plastic (e.g., polypropylene, polycarbonate, polystyrene, polyethylene), or any other elastic material capable of creating a fluidic seal.

The gasket 740 may have a shape corresponding to the shape of the lid. Accordingly, for example, the gasket may have a rectangular shape. In other variations, the gasket may be square, circular, trapezoidal, or any other shape that allows for a seal to be formed between the opening of the container body and the lid. As illustrated in FIGS. 7A-C, the gasket 740 may comprise a first pair of linear segments opposite each other, and a second pair of linear segments opposite each other. A length associated with each of the first and second pairs may be different. In some variations, the length each linear segment of the first pair may be about 1 inch to about 2 inches, about 1 inch to about 3 inches, about 1 inch to about 4 inches, about 1 inch to about 5 inches, or about 1 inch to about 6 inches. In an exemplary variation, the length may be about 3.9 inches. In some variations, the length of each linear segment of the second pair may be about 0.5 inches to about 1 inch, about 0.5 inches to about 2 inches, about 0.5 inches to about 3 inches, about 0.5 inches to about 4 inches, about 0.5 inches to about 5 inches, or about 0.5 inches to about 6 inches. In an exemplary variation, the width may be about 0.6 inches. A width associated with each segment of the first and second pairs may be the same. In some variations, the width of each segment may be about 0.1 inches to about 0.5 inches, such as about 0.15 inches. A thickness associated with each segment of the first and second pairs may be the same. In exemplary variation, the thickness may be about 0.01 inches to about 0.1 inches, such as about 0.06 inches. Each segment of the first pair may be connected to each segment of the second pair. The connection may comprise an inner radius of curvature associated with an inner edge of the segments and an outer radius of curvature associated with an outer edge of the segments. The outer radius of curvature may be greater than the inner radius of curvature.

In some variations, the fluidic sealing component (e.g., a gasket) may be impinged upon by a lip or other raised section of the lid to couple the fluidic sealing component to thereto. In this way, a sealing pressure on the fluidic sealing component may be increased.

In some variations, the fluidic sealing component may be integrally formed or fixedly attached to the lid. For example, referring again to FIG. 21B, the lid 2130 may include an extension 2132 extending distally from a bottom of the lid 2130 and configured to surround at least a portion of an exterior of the annular container body 2120, such as a proximal portion of the annular container body 2120. The extension 2132 may prevent fluid within cavity 2125 of the body 2120 from escaping to an external environment. In some variations, the lid 2130 may include a plurality of fluidic sealing components, such as the extension 2132 and a gasket (not shown) having a shape corresponding to the annular shape of the lid 2130. The gasket may be circular, ovular, or another annular shape. A width or diameter of the gasket may be about 1 inch to about 6 inches (including all ranges and subranges in between).

### d. Pressure Management Feature

The lid of the deactivation container described herein may comprise a pressure management feature to allow fluid (e.g., gas) to flow from within the deactivation container to an external environment. In some cases, the pressure management feature may additionally limit a fluid transfer rate between the deactivation container and the external environment (e.g., from a cavity defined by the deactivation container to the external environment). In some variations, the pressure management feature may include a vent within the lid, and may further include a membrane aligned with the vent and configured to control (e.g., via a pore size thereof) permeation of liquids and/or gases from the cavity to the external environment via the vent. In alternate variations, the pressure management feature may include a valve, which may be a one-way valve or, in some variations, a two-way valve, within the lid. The lid with a one-way valve, which may be a pressure relief valve, may allow fluid to flow from within the cavity to the external environment. For example, a gaseous byproduct of an exothermic reaction between an alkali metal device and an alkali metal reactant contained within cavity may flow through the pressure management feature. By providing a path for the gaseous byproduct to escape the cavity, the pressure management feature (e.g., vent, valve) may relieve pressure buildup within the cavity resulting from the chemical reaction.

As the pressure management feature is configured to regulate fluid transfer out of the cavity, the pressure management feature may be utilized while the lid is in the closed position (e.g., when the lid is engaged with the support and/or the container body). When the lid is in the closed position, the pressure management feature itself may have a closed configuration and an open configuration. With the pressure management feature in the closed position, no liquid may flow therethrough. In some variations, the pressure management feature may be maintained in the closed position by a security mechanism releasably coupled to one or more of the pressure management feature, the lid, the support, and the body. When the pressure management feature includes a vent, an obstruction of the vent via the security mechanism may define the closed position. Oppositely, the pressure management feature may be in the open position when the security mechanism is removed therefrom. In the open position, the pressure management feature may have a pressure rating of about 0.5 psi to about 20 psi, such as about 0.6 psi to about 15 psi, about 0.7 psi to about 10 psi, about 0.8 psi to about 5 psi, about 0.9 psi to about 4 psi, about 1 psi to about 3 psi, about 1.25 psi to about 2.75 psi, about 1.5 psi to about 2.5 psi, or about 1.75 psi to about 2.25 psi (including all ranges and subranges therebetween). For example, the pressure rating may be about 1 psi to about 2 psi.

In some variations, the lid may include more than one pressure management feature to facilitate faster release of the gaseous byproduct to the external environment. In another variation, the pressure management feature, such as the two-way valve, may be configured to facilitate fluid from the external environment into the deactivation container. For example, a user may first place the device into the deactivation container, close the lid, and pour a fluid (e.g., alkali metal reactant or alkali metal solubilizer) through the pressure management device and into the cavity thereof. In a further variation, the lid may comprise a one-way valve as described previously and a separate fluid port configured to facilitate fluid transfer from the external environment into the deactivation container. In this way, a gaseous byproduct of a chemical reaction between the fluid and the device may be released via the one-way valve at the same time that the fluid is poured through the fluid port.

As introduced above with reference to FIGS. 22A and 22C, the pressure management feature may include a membrane vent. The vent may extend transversely through the lid 2200. The vent may have a first opening 2236a at a top of the lid 2200, a lumen (not shown) extending through the lid 2200, and a second opening 2236b at a bottom of the lid. The vent (e.g., the first and second openings 2236a,b and the lumen) may have a maximum dimension (e.g., width, diameter, major axis) of about 0.01 mm to about 10 mm, such as about 0.025 mm to about 5 mm, about 0.5 mm to about 4 mm, about 0.75 mm to about 3 mm, about 1 mm to about 2.75 mm, about 1.25 mm to about 2.5 mm, about 1.5 mm to about 2.25 mm, or about 1.75 mm to about 2 mm (including all ranges and subranges therebetween). For example, the vent may have a maximum dimension of about 0.01 mm to about 2 mm. The membrane vent may further include a membrane 2238 configured to cover the second opening 2236. For example, the bottom of the lid may include a recess 2237 therein, the membrane being positionable within the recess 2237, and the recess 2237 being sized and shaped to maintain the membrane 2238 adjacent to or against the second opening 2236. That is, the recess 2237 may have a dimension that is about equal to or larger than a dimension of the membrane 2238 in order to hold the membrane therein. In some variations, the membrane may be coupled to the recess via an adhesive (e.g., glue). Generally, the membrane 2238 may have a maximum dimension (e.g., width, diameter, major axis) that is about equal to or larger than a maximum dimension (e.g., width, diameter, or major axis) of the second opening 2236b such that the membrane 2238 may completely cover the second opening 2236b. In some variations, the membrane 2238 may have a maximum dimension of about 0.25 cm to about 2 cm, such as about 0.5 cm to about 1.75 cm, about 0.75 cm to about 1.5 cm, or about 1 cm to about 1.25 cm. For example, the width or diameter of the member 2238 may be about 1 cm. Moreover, the recess 2237 may have a shape that is similar to or substantially the same as the membrane 2238. For example, the recess 2237 and the membrane 2238 may each have a circular shape, an ovular shape, a rectangular shape, a square shape, a triangular shape, an irregular shape, or another suitable shape. In some variations, the membrane may extend at least partially within the lumen of the vent.

The membrane 2238 may be configured to control (e.g., limit) fluid exchange between the container body cavity and an environment external to the first opening 2236a. In some variations, the membrane 2238 may have a pore size that allows the gaseous byproduct (e.g., hydrogen) to permeate therethrough but does not allow the liquid within the cavity to permeate therethrough up to a predetermined pressure. For example, the membrane may prevent liquid from spilling from the cavity if the deactivation container was inverted. The membrane pore size may be about 0.00001 μm to about 50 μm, such as about 0.0001 μm to about 45 μm, about 0.001 μm to about 40 μm, about 0.01 μm to about 35 μm, about 0.1 μm to about 30 μm, about 1 μm to about 25 μm, about 5 μm to about 20 μm, or about 10 μm to about 15 μm (including all ranges and subranges in between). For example, the pore size may be about 0.02 μm to about 15 μm, such as about 0.2 μm to about 0.5 μm. The membrane 2238 may be fabricated with a desired pore size using materials such as polymers, including but not limited to one or more of polytetrafluoroethylene (PTFE), expanded PTFE, sintered PTFE, PTFE-based compositions (e.g., Teflon), polychlorotrifluoroethylene (PCTFE), fluorinated ethylene propylene (FEP), and the like. The membrane 2238 may have a pressure rating (e.g., a predetermined pressure rating) that allows gaseous byproduct to permeate therethrough before the pressure buildup within the cavity causes damage to the deactivation container. The pressure rating may be about 1 psi to about 16 psi, such as about 2 psi to about 14 psi, about 3 psi to about 12 psi, about 4 psi to about 10 psi, or about 5 psi to about 8 psi (including all ranges and subranges therebetween).

Referring back to FIG. 2, the lid 230 may comprise an alternative pressure management feature, pressure relief valve 236, configured to release gas from the container. As discussed in more detail herein, a reaction between an alkali metal device and a solubilizer within the container may result in generation of a gas, which may increase the pressure inside of the container during a deactivation process. Accordingly, to ensure safe deactivation, the pressure relief valve 236 may be within the lid as doing so may advantageously release gas (i.e., a chemical reaction byproduct) from within the cavity while safely retaining all liquid as well as the device contained therein. This configuration may prevent pressure build-up of a gas and unintentional spills or leaks of a liquid during transportation and/or use, particularly when the security mechanism may not be coupled to the lid 230 and/or pressure relief valve 236. Additionally, the upright orientation of the deactivation container described herein may further prevent unintentional leaks through the pressure relief valve 236 during, for example, sloshing of the liquid (e.g., a solution containing alkali metal reactant or solubilizer), which may otherwise occur if the pressure relief valve 236 is positioned on a sidewall of the deactivation container.

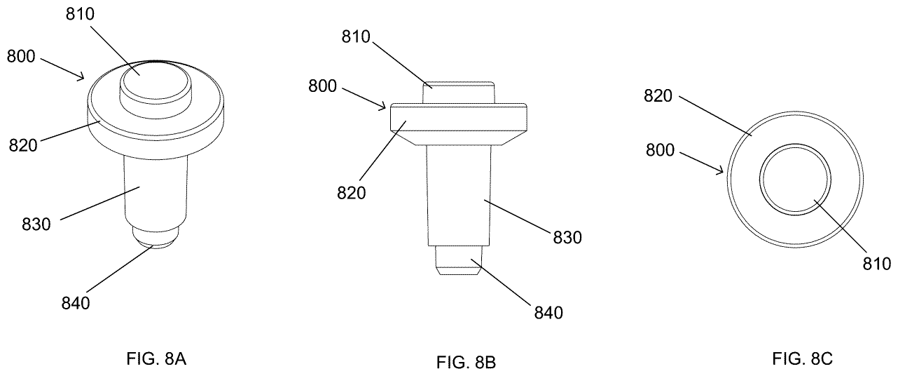

FIGS. 8A-C illustrate an exemplary variation of a pressure relief valve 800 for use in the deactivation containers described herein. The pressure relief valve 800 may be configured to direct fluid flow therethrough. The pressure relief valve 800 may comprise a first portion 810 and a second portion 820. A surface of the first portion 810 may be configured to be flush with a top surface of a lid or may be configured to extend from the lid. The second portion 820 may be configured to be received within by, for example, a valve receiver of a lid. The first portion 810 may comprise a first diameter and the second portion 820 may comprise a second diameter that is greater than the first diameter. The first and/or second diameters may be determined by one or more of a fluid flow rate, a fluid pressure, a fluid volume, and a fluid temperature. For example, an increase in a desired fluid flow rate may correspond to an increase in the first and/or second diameter. In some variations, the first diameter may be about 0.1 inches to about 0.3 inches, about 0.1 inches to about 0.4 inches, or about 0.1 inches to about 0.5 inches. In some variations, the second diameter may be about 0.1 inches to about 0.4 inches, about 0.1 inches to about 0.5 inches, or about 0.3 inches to about 0.4 inches. The pressure relief valve 800 may further comprise a third portion 830 and a fourth portion 840. The third portion 830 may be received within an opening of the lid. The third portion 830 may comprise a length sufficient to extend from a top of the lid to a bottom of the lid. In an exemplary variation, the length of the third portion may be about 0.1 inches to 1 inch, such as about 0.33 inches. The third portion 830 may comprise a third diameter and the fourth portion 840 may comprise a fourth diameter that is less than the third diameter. In some variations, the third diameter may be about 0.1 inches to about 0.2 inches, about 0.1 inches to about 0.3 inches, about 0.1 inches to about 0.4 inches, or about 0.1 inches to about 0.5 inches. In some variations, the fourth diameter may be about 0.1 inches to about 0.2 inches, about 0.1 inches to about 0.3 inches, about 0.1 inches to about 0.4 inches, or about 0.1 inches to about 0.5 inches. The second diameter may be greater than each of the third and fourth diameters. In an exemplary variation, a length of the fourth portion may be about 0.01 inches to about 0.5 inches, such as about 0.08 inches. A surface of the fourth portion 840 may be configured to be flush with the bottom of the lid or may be configured to contact a retention clip. In some variations, a surface of the fourth portion 840 may also be configured to contact a fluid.

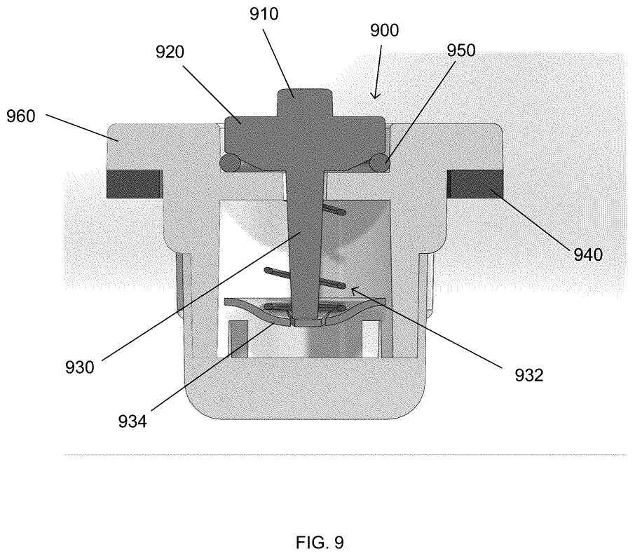

The pressure relief valve may further comprise one or more elastic components configured to create a fluidic seal. FIG. 9 illustrates an exemplary variation of a pressure relief valve 900 received within a lid 960. The pressure relief valve 900 may comprise a first portion 910, a second portion 920, and a third portion 930. The first portion 910 may comprise a first diameter, the second portion 920 may comprise a second diameter greater than the first diameter, and the third portion 930 may comprise a third diameter less than each of the first and second diameters. The range of diameters for each portion may be similar to those described for the variation illustrated in FIG. 8. In contact with the second portion 920 may be a sealing mechanism 950. The sealing mechanism 950 may comprise a deformable material configured to create a fluidic seal. The sealing mechanism 950 may extend along a perimeter of the second portion 920 when the second portion 920 is received with a valve receiver of the lid 960. For example, the sealing mechanism 950 may comprise rubber, plastic, a polymer, or any other elastic material suitable for sealing. In an exemplary variation, the sealing mechanism may comprise a circular component such as an O-ring. In one variation in which the sealing mechanism comprises an O-ring, an outer diameter of the O-ring may be about 1 mm to about 20 mm, about 5 mm to about 15 mm, or about 9 mm to about 11 mm, such as about 10 mm. In some variations, the sealing mechanism may comprise a width about 0.5 mm to about 4 mm, such as about 1 mm. In some variations, the sealing mechanism may comprise a durometer configured to create a fluidic seal upon compression between two surfaces (as in when a pressure relief valve is received within the lid). The durometer may be about 0 A to about 60 A, such as about 50 A. Also shown in FIG. 9 is a gasket 940 coupled to a bottom surface of the lid 960. The gasket 960 may extend along a perimeter of the lid 960 such that a fluid seal is created when the lid 960 is engaged with a container body (not shown).

The pressure relief valve may further comprise one or more elastic components configured to transition the pressure relief valve from an open position to a closed position (or vice versa). For example, the pressure relief valve 900 may comprise a retention ring 934 and a spring 932. The retention ring 934 may be configured to apply an elastic force to the third portion 930 and/or the spring 932. Accordingly, the retention ring 934 may comprise a material configured to elastically deform or otherwise provide a biasing force upon deflection (as in response to a pressure applied by a fluid within the deactivation container). The retention ring 934 may comprise stainless steel, copper, or aluminum. The retention ring 934 may be operatively coupled to each of the spring 932 and third portion 930. The retention ring 934 may comprise a circular body with a central opening corresponding to the third portion 930 of a pressure relief valve. In some variations, an outer diameter of the retention ring 934 may be about 0.1 inches to about 0.7 inches, such as about 0.375 inches. An inner diameter associated with the central opening of the retention ring 934 may be about 0.05 inches to about 0.4 inches, such as about 0.125 inches.

The pressure relief valve described herein may be configured to transition from a closed position to an open position when a force is applied to the retention ring. Accordingly, an amount of force applied by the retention ring 934 to the third portion 930 may be determined by a pressure created as a byproduct of a reaction between an alkali metal and an alkali metal reactant or solubilizer contained within the container body. The pressure force may be sufficient to cause the pressure relief valve 900 to transition from a closed position to an open position. The transition may occur based on a predetermined pressure differential threshold. The pressure differential may be based on the relative values of a cavity pressure within the cavity of the container body and an environmental pressure external to the container body. For example, the pressure relief valve 900 (or any other variation of a pressure relief valve described herein) may open based on a predetermined threshold about 0.1 psi to about 10 psi (including all values and sub-ranges therein). In other variations, the predetermined threshold may be about 0.5 psi to about 2 psi, about 0.5 psi to about 5 psi, about 0.5 psi to about 10 psi, about 0.5 psi to about 15 psi, about 0.5 psi to about 20 psi, or about 0.5 psi to about 25 psi.

The spring 932 may be configured to resist the force applied by the pressure and/or retention ring 934. In this way, the spring 932 may transition from the open position to the closed position if the pressure differential falls below the predetermined threshold. The spring 932 may comprise one or more of stainless steel, aluminum, copper, and silver. The spring may have a number of coils, thickness, and outer diameter corresponding to an expected force profile. In some variations, the spring may comprise between 1 coil and 20 coils, or between 10 coil and 15 coils, such as about 11.5 coils. In some variations, the wire diameter may be about 0.01 inches to about 0.1 inches, such as about 0.02 inches. In some variations, the spring 932 may comprise an outer diameter about 0.1 inches to about 0.5 inches, such as about 0.24 inches. Accordingly, in an exemplary variation, the spring 932 may be configured to depress according to a predicted pressure applied by the byproduct of the reaction between alkali metal of an alkali metal device and an alkali metal reactant contained within the deactivation container described herein. In some variations, the spring 932 may be configured to depress at a rate about 0.1 lbf per inch to about 2 lbf per inch.

In some variations, the pressure management feature may include an umbrella valve or a Belleville valve. As an example, like the pressure relief valve described above, the umbrella valve may include a one-way elastomeric valve, but may further include a sealing disk having an umbrella or diaphragm shape that may sit on the lid. When subjected to a pressure gradient, the sealing disk may flex open to relieve the pressure buildup. An umbrella valve of the lids herein may be configured to flex open in response to pressure within the deactivation container of about 0.5 psi to about 20 psi, such as about 0.6 psi to about 15 psi, about 0.7 psi to about 10 psi, about 0.8 psi to about 5 psi, about 0.9 psi to about 4 psi, about 1 psi to about 3 psi, about 1.25 psi to about 2.75 psi, about 1.5 psi to about 2.5 psi, or about 1.75 psi to about 2.25 psi (including all ranges and subranges therebetween). For example, the umbrella valve may flex open in response to a pressure of about 1 psi to about 2 psi.

### e. Security Mechanism

The lid of the deactivation container may comprise a security mechanism configured to maintain a closed position of, or otherwise obstruct, the pressure management coupled to, or defined by, the lid. Maintaining the closed and/or obstructed position of the pressure management feature before and/or after a deactivation process may avoid unintentional spills or leaks, as in the event of an unexpected inversion (e.g., tipping of the deactivation container). For example, fluid may unintentionally flow through the pressure management feature upon an unexpected inversion if the pressure management feature is allowed to transition to an open position and/or is unobstructed. Additionally, the security mechanism may keep the pressure management feature (e.g., valve or vent) closed during transportation of the deactivation container in an airplane or ground transport over mountain passes, where the pressure differential between the pressure within the cavity of the deactivation container (which may contain a fluid) and the external pressure (e.g., cargo pressure) may be sufficient to cause the pressure management feature to allow gas to escape from the cavity, if not for the security mechanism keeping the pressure management feature closed and/or obstructed. In some variations, the security mechanism may advantageously indicate whether or not the deactivation container and/or pressure management feature has been tampered with by an unauthorized user. For example, a partially or completely removed security mechanism may indicate unauthorized tampering of the deactivation container.

The security mechanism may be configured to releasably couple to the lid, and optionally the body and/or support, of the deactivation kit such that it may be removed by a user prior to use of the deactivation container in a deactivation process. In one example, the security mechanism may include a clip, a band, and/or an adhesive (e.g., tape). The security mechanism may be configured to cover a pressure management feature of one or more container lids of a deactivation kit. For example, in some variations, as shown in FIG. 20, a single security mechanism 2035 may cover the pressure management feature (not shown) of each lid 2030 (e.g., two or more) of the deactivation kit 2000. In other variations, each lid may utilize its own security mechanism 2035 to engage and/or obstruct the pressure management feature. Additionally, or alternatively, a security mechanism 2035 may releasably couple to one or more sidewalls of each container 2015 of the deactivation kit 2000, such as to a first external sidewall 2022 of the container body 2020.

In another exemplary variation, the security mechanism may comprise a cover with one or more perforations. The one or more perforations may allow a user to separate one or more sections of the security mechanism from the deactivation container such that the pressure relief valve of the lid may be opened to allow fluid to flow therethrough. For example, referring back to FIG. 2, a security mechanism 240 may comprise a first section 241 separated from a second section 243 by a first perforated portion 245, and a third section (not shown) separated from the second section 243 by a second perforated portion (not shown). Accordingly, the first section 241, second section 243, and third section may form a strip, where the first perforated portion 245 and second perforated portion are configured across the strip. The second section 243 may be separable from the first and third sections along the first and second perforated portions. For example, a user may insert their finger into the indent 234 such that the user's finger can be placed underneath the second section 243. The user may then apply a force to the second section 243 sufficient to tear away the second section 243 along the first perforated portion 245 and second perforated portion. Separating the second section from at least one of the first and third sections may disengage the security mechanism from the lid and/or pressure relief valve, thereby allowing the pressure relief valve to open when a pressure differential meets or exceeds a predetermined threshold.

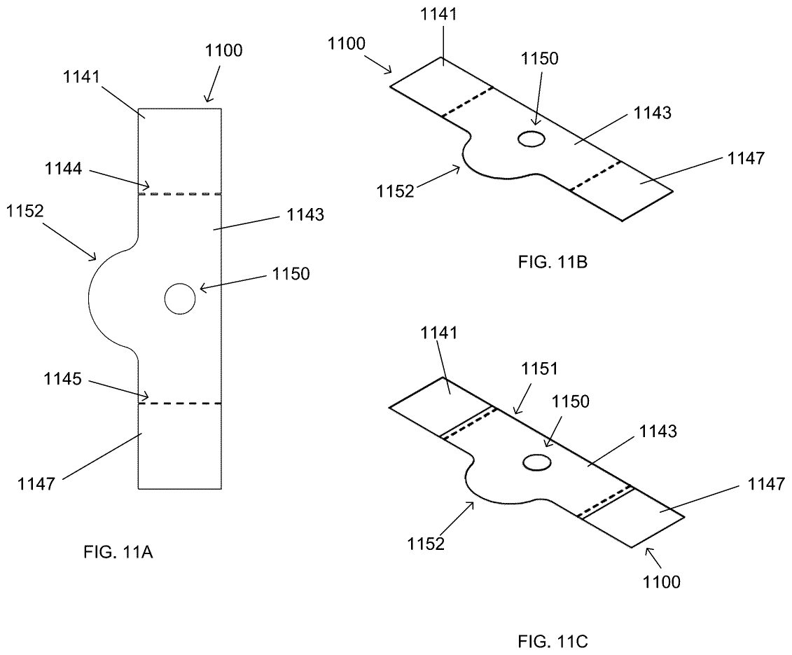

A further variation of a security release mechanism comprising a cover with one or more perforations is shown in FIGS. 11A-11C. Like the variation shown in FIG. 2, the cover 1100 may comprise a first section 1141 separated from a second section 1143 by a first perforated portion 1144, and a third section 1147 separated from the second section 1143 by a second perforated portion 1145. The second section 1143 may comprise an opening 1150 that is configured to receive a portion of a pressure relief valve (not shown), which may enable the second section 1143 to couple directly to each of the pressure relief valve and the lid and optionally the body of the deactivation container. The second section 1143 may further comprise a semi-circular extension 1152, which may be shaped such that a user may easily lift or otherwise move (e.g., using a finger or other tool) the second section 1143 relative to the first and third section 1141, 1147 to separate the second section 1143 from the first and third sections 1141 1147 along the first and second perforated portions 1144, 1145 respectively. In this manner, the cover 1100 may be released from the pressure relief valve. While depicted as semi-circular, the extension 1152 may have any shape suitable to allow a user to easily access and/or grasp the second section 1143 while engaged with the pressure relief valve, such as, for example, square, rectangular, semi-ovular, triangular, or the like. Additionally, in some variations, the cover 1100 may further comprise an adhesive layer 1151 on a container-facing surface of the cover 1100 that may be configured to releasably adhere the cover 1100 to the pressure relief valve, the lid, and/or one or more of the deactivation container sidewalls. The adhesive layer 1151 may be on the second section 1143 only, or may cover at least a portion of the second section 1143 and at least a portion of one or more of the first section 1141 and the third section 1147. For example, as can be seen best in FIG. 11C, in some variations the adhesive layer 1151 may extend across the width of the second section 1143 and may cover a portion of each of the first section 1141 and third section 1147.

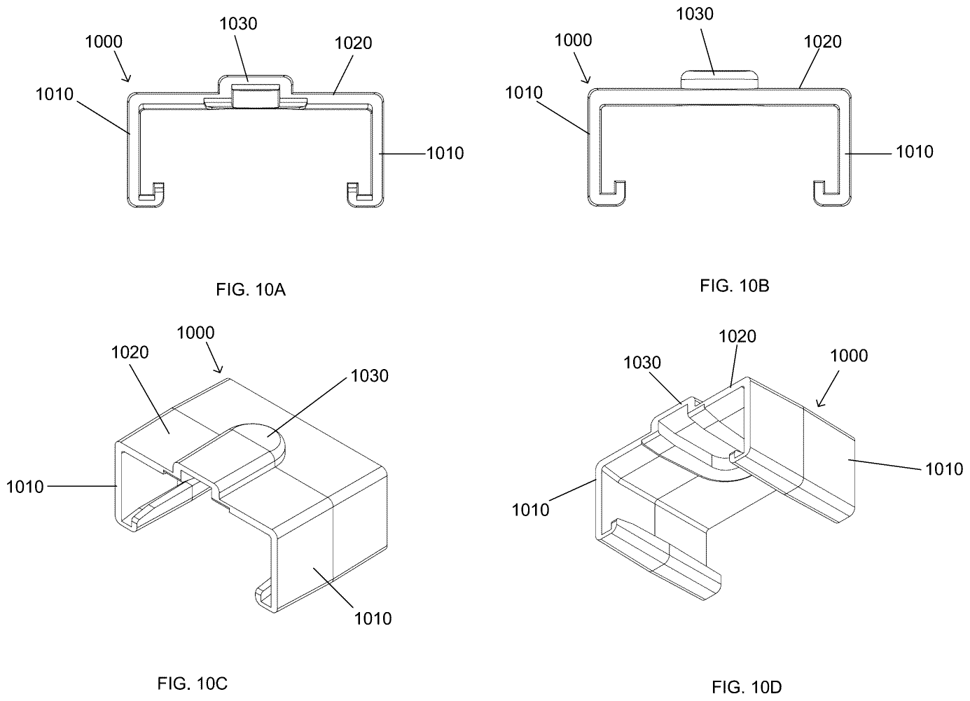

FIGS. 10A-10D illustrate an alternative variation of a security mechanism 1000. The security mechanism 1000 may comprise a clip having a central portion 1020 and a side portion 1010 extending therefrom. The central portion 1020 of the security mechanism 1000 may comprise a valve cover 1030 configured to be positioned over at least a portion of the valve. The valve cover 1030 may prevent the pressure relief valve from transitioning from a closed position to an open position, even when a pressure differential meets or exceeds a predetermined threshold. The valve cover 1030 may comprise a shape corresponding to a shape of the pressure relief valve. In some variations, the security mechanism 1000 may comprise a plurality of side portions 1010. The side portions 1010 may each extend from the central portion 1020. For example, each side portion 1010 may extend from a different (e.g., opposite) side of the central portion 1020. Each side portion 1010 may be configured to releasably couple to a container body sidewall. The side portion 1010 may include a hook or other curved portion that corresponds to or otherwise configured to engage an edge or another portion or feature of the container body sidewall. The security mechanism 1000 may rely on a friction force to maintain its position over the pressure relief valve. The security mechanism 1000 may be configured to slide off the lid such that the valve 1030 is no longer in contact with the pressure relief valve, thereby allowing the pressure relief valve to open when a pressure differential meets or exceeds a predetermined threshold.

The security mechanism 1000 may have dimensions corresponding to dimensions of the deactivation container described herein. For example, the central portion 1020 may comprise a width corresponding to a width of the container body and/or container body lid. In some variations, the central portion width may be about 0.5 inches to about 1 inch, about 0.5 inches to about 1.5 inches, or about 0.5 inches to about 2 inches. In some variations, the side portion height may be about 0.1 inches to about 0.5 inches, about 0.1 inches to about 1 inch, or about 0.35 inches to about 0.45 inches. The security mechanism 1000 may comprise a length. In some variations, the security mechanism length may be about 0.5 inches to about 1 inch, about 0.5 inches to about 1.5 inches, 0.5 inches to about 2 inches, about 1 inch to about 1.5 inches, about 1 inch to about 2 inches, or about 1.5 inches to about 2 inches.

Additionally, or alternatively, the security mechanism may comprise an adhesive, a latch, a band, a zip-tie, or other mechanical fastening means. For example, the security mechanism may comprise a hook, where a first end of the hook is engaged with a sidewall of the body and a second end of the hook is engaged with the valve (e.g., via an opening (e.g., through hole, depression, indent, groove) of the valve). The hook may be removed by a user by removing an end of hook from the opening (e.g., by pulling the hook away from valve). In another variation, the security mechanism may comprise a band that is configured to encompasses at least a portion of the deactivation container. For example, the band may be releasably coupled to the lid and/or at least one sidewall of the body. In yet another variation, the band may be configured to encompass the entire vertical dimension (i.e., height) of the body. The band may be positioned such that it covers a portion of the valve, thereby preventing the pressure management feature from transitioning from a closed position to an open position. The band may be removed by a user by sliding or pulling the band away from the surface of one or more the body and the valve.

The security mechanism may comprise any material suitable for releasably attaching to the deactivation container and/or lid thereof. For example, the security mechanism may comprise plastic (e.g., polypropylene, polycarbonate, polystyrene, polyethylene), a metal (e.g., stainless steel, steel, aluminum, copper), a rubber (e.g., nitrile, neoprene, ethylene propylene, silicone, fluorocarbon), or combination thereof.

### f. Support

As discussed above, the deactivation kits herein may comprise one or more supports, such as one support or a plurality thereof (e.g., two, three, four, or more). The support may be configured to extend from, receive, and/or couple to one or more deactivation containers. In some variations, the support may be integrally formed with one or more deactivation containers. In this way, the support may maintain the deactivation container(s) in an upright position, which may prevent accidental spills or leaks of any fluid contained therein, facilitate fluid flow around a device received therein, facilitate a substantially uniform reaction between a device (e.g., an alkali metal device) and the fluid (e.g., alkali metal reactant), and facilitate flow of a gas through the pressure management device of the lid of the deactivation container.

In some variations, the support may include a bridge situated between and configured to couple two deactivation containers. The bridge may couple the containers at corresponding portions thereof, such as at a top or proximal portion (e.g., adjacent an opening of each of the containers), at a midportion, or at a distal portion (e.g., adjacent a bottom side or surface of each of the containers) thereof. In some variations, the support may include a plurality of bridges, where each of the plurality of bridges is coupled to a different portion (e.g., proximal, mid, or distal) of the containers or to the same portion (proximal, mid, distal) but at a different location along a longitudinal axis of the container. The bridge may include a plate or crossbeam having a first side coupled to a sidewall of a first deactivation container and a second, opposite side coupled to a sidewall of a second deactivation container. The sidewalls of the first and second deactivation containers may face each other. The bridge may have a longitudinal axis that is parallel or substantially parallel to a longitudinal axis of an opening of each of the containers, or, conversely, that is transverse (e.g., perpendicular or substantially perpendicular) to a longitudinal axis of a body of each of the containers. In some variations, the bridge may be part of a frame having one or more openings shaped to receive a deactivation container therein. The frame may surround an external perimeter of the deactivation container, and may be shaped and sized to be positioned along, and sit flush with, a predetermined portion of the container body, such as at a proximal portion thereof.

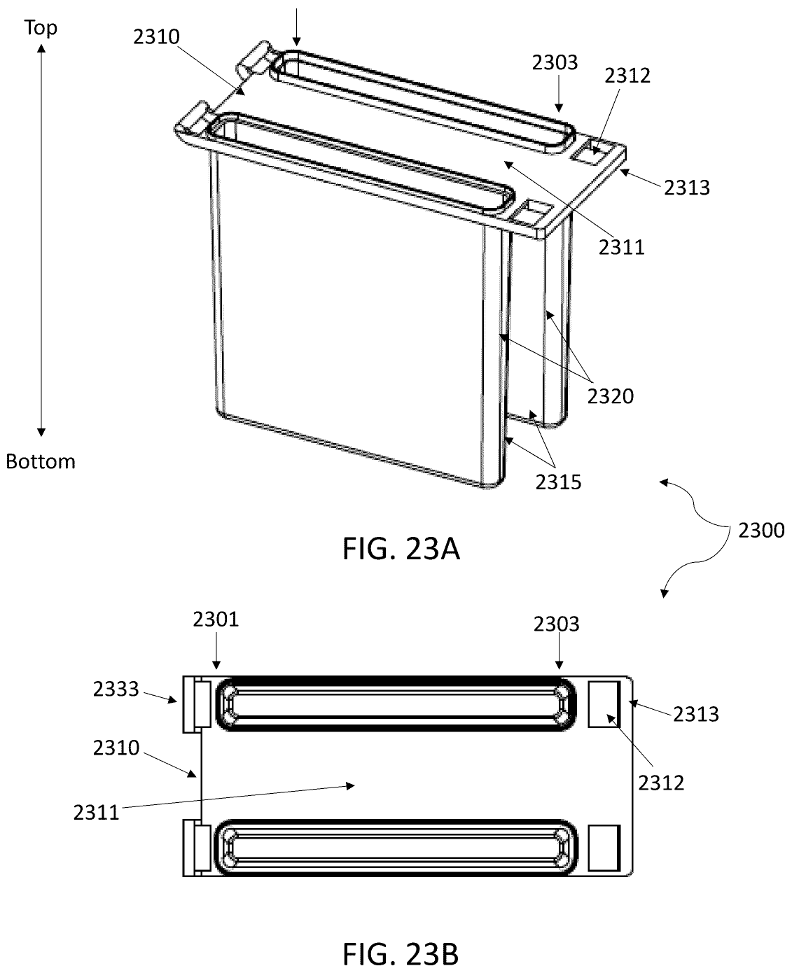

An exemplary deactivation kit (or system) 2300, having deactivation containers 2315 coupled via a support, bridge 2010, is illustrated in FIGS. 23A and 23B. As shown, the bridge 2010 may be positioned at a top or proximal portion of each of the containers 2315, where it may maintain bodies 2320 of the containers 2315 in a fixed relative position. That is, the bridge 2010 may stabilize each of the containers 2315 so that each container may act as a leg to maintain the deactivation kit 2300 in an upright position.

In particular, the bridge 2010 may include a central plate 2011 that is coupled to an inner surface or sidewall of a body 2320 of each of the containers 2315. In some variations, the central plate 2011 may include a crossbeam configured to increase a stiffness of the central plate 2011 and bolster its ability to stabilize the deactivation kit 2300. In other variations, the bridge 2010 may comprise both a plate (e.g., central plate) and one or more crossbeams spaced apart from the plate (e.g., longitudinally or along a height of the deactivation container). The central plate 2011 may be rigid or substantially rigid such that it is configured to maintain a gap between the containers of about 1 cm to about 20 cm, such as about 2 cm to about 18 cm, about 3 cm to about 17 cm, about 4 cm to about 16 cm, about 5 cm to about 15 cm, about 6 cm to about 14 cm, about 7 cm to about 13 cm, about 8 cm to about 12 cm, or about 9 cm to about 11 cm (including all ranges and subranges therebetween). The gap between the containers 2315 may help the deactivation kit 2300 to balance on its two points of contact, the bottoms of the bodies 2320. In some variations, the bridge 2310 may be part of a frame that surrounds an external perimeter of one or both of the container bodies 2320. The frame may be shaped and sized to be positioned along, and sit flush with, a predetermined portion of a container body, such as at a proximal portion thereof (as in FIG. 23A).

As described in detail above with reference to FIG. 20 and FIG. 22A, in some variations, the support may include one or more engagement features for coupling to a lid. The support may, in some cases, include one or more engagement features for coupling to each of a plurality of lids (e.g., at least two lids). Referring again to FIG. 23A and to FIG. 23B, the support, bridge 2310, may extend longitudinally beyond first and second ends 2301,2303 of the bodies 2320. Adjacent to the first end 2301, the bridge 2310 may include (e.g., may be integrally formed with) rods 2333. The rods 2333 may be configured to rotatably couple to a lid via a hinge clip. Adjacent the second end 2303, the bridge 2310 may include (e.g., may be integrally formed with) an opening 2312 configured to receive at least a portion of a lock (e.g., a latch) of the lid. The opening 2312 may be defined by sidewalls, one or more of which and may be configured to engage a “stop” element on the lock (e.g., a ledge or protrusion thereof) to prevent the lid from decoupling from the body in the closed position. More specifically, in the closed position, the lock (not shown) may be configured to releasably engage a bottom surface of a sidewall 2313 that frames the opening 2312. The lock may have an at-rest configuration that bias a “stop” element of the lock (e.g., a ledge) toward and against the bottom surface of sidewall 2313, thereby engaging the lock with the bridge 2310 and preventing the lock, and thus the lid coupled thereto, from moving or otherwise deviating from the intended closed position. To disengage the lock from the bridge 2310, the lock may be configured to be transitioned to a flexed configuration that overcomes the bias of the lock and moves the “stop” element thereof toward the opening 2312 and thus out from under the sidewall 2313.

Moreover, in some variations, the support may be a base comprising an opening and a cavity. Each of the opening and cavity of the base may be configured to receive one or more deactivation containers before, during, and/or after a deactivation process (e.g., deactivation of an alkali metal of an alkali metal device). The opening may comprise a shape corresponding to a shape of the deactivation container. Additionally, the cavity may comprise a width, length, and depth corresponding to the shape and/or size of the deactivation container. In this way, the base may partially or completely surround the one or more deactivation containers.

Accordingly, the base may comprise a shape corresponding to one or more deactivation containers, such as a box, a cube, a rectangular prism, a cylinder, a sphere, a pyramid, or combination thereof. In an exemplary variation, the base may comprise a chamber (i.e., cavity) formed by a bottom, a first pair of sidewalls, and a second pair of sidewalls. In some variations, one or more standoff members may extend from, or be coupled to, one or more of the sidewalls. The first pair of sidewalls may comprise a first and a second sidewall. The first and second sidewalls may be attached to opposite ends of the bottom. The first and second sidewalls may be parallel to one another. The second pair of sidewalls may comprise a third and a fourth sidewall. The third and fourth sidewalls may be attached to opposite ends of the bottom. The third and fourth sidewalls may be parallel to one another. Each sidewall of the base may be substantially planar or may be curved (e.g., concave, convex). In some instances, it may be advantageous to utilize a base with substantially planar sidewalls as this may allow for easier packing, shipping, and/or storage of the base (e.g., with one or more deactivation containers therein) The height of the base may be less than the height of the deactivation container such that the deactivation container may be securely maintained in an upright position and/or may be easily accessed while coupled to (e.g., received in) the base. For example, the height of at least one sidewall of the base (e.g., all sidewalls) may be less than the height of the deactivation container.

The support may be of a material suitable to support the mass of the deactivation container, device, and fluid described herein while minimizing heat transfer to the user and reducing shipping and/or packaging considerations. Accordingly, the support may be made of a paper (e.g., cardboard), a metal (e.g., stainless steel, steel, aluminum, copper), a plastic (e.g., polypropylene, polycarbonate, polystyrene, polyethylene), or combination thereof. For example, a support (e.g., a bridge) may be manufactured using one or more of the same materials used to manufacture a deactivation container body. In some variations, the support may be integrally formed with, or fixedly attached to, one or more deactivation container bodies. As another example, a base may be manufactured using a flat sheet of cardboard. The cardboard may be cut to according to a predetermined configuration. Then, the cardboard may be folded one or more times to create a support (e.g., a base) comprising a three-dimensional shape. In this way, the support may be shipped as a flat piece of cardboard and assembled upon delivery to, for example, a clinical setting.

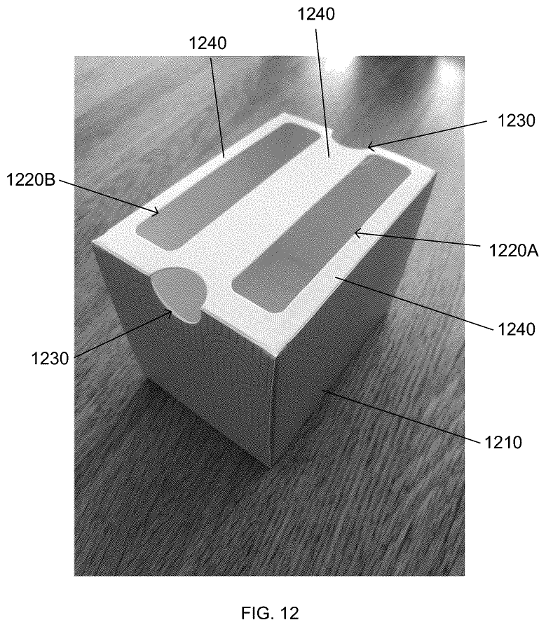

FIG. 12 shows an illustrative variation of a base 1210. The base 1210 may comprise a closed bottom and a standoff member 1240 coplanar with a top of the base 1210, as defined by the upper edges of the sidewalls. In some variations, the base 1210 may comprise a plurality of standoff members. The standoff member 1240 may be configured to maintain a gap between the deactivation container and a sidewall of the base and/or between two or more deactivation containers. Each of these gaps may advantageously form a pocket of air that may reduce, inhibit, or otherwise minimize heat transfer from a deactivation container to a sidewall of the base 1240 and/or between two or more deactivation containers. For example, the base may be configured such that at least one sidewall, and preferably each sidewall, of the base may be maintained below about 50 degrees C. during and/or after a deactivation process within a deactivation container coupled to (e.g., received within) the base. In other variations, at least one sidewall, and preferably each sidewall, may be maintained at a temperature of about 30 degrees C. to about 60 degrees C., 30 degrees C. to about 55 degrees C., 30 degrees C. to about 50 degrees C., about 40 degrees C. to about 60 degrees C., about 40 degrees C. to about 55 degrees C., about 40 degrees C. to about 50 degrees C., or about 45 degrees C. to about 55 degrees C. This may advantageously allow a user to handle the base during and/or after an exothermic reaction.

The standoff member 1240 may comprise one or more openings 1220 at the top of the base that are each configured to receive a deactivation container. In some variations, the standoff member 1240 may comprise a plurality of openings (e.g., two, three, four, or more). As shown in FIG. 11, the standoff member 1240 may comprise a first opening 1220A and a second opening 1220B. The first opening 1220A may comprise a shape corresponding to a shape of a first deactivation container and the second opening 1220B may comprise a shape corresponding to a shape of a second deactivation container. Each of the plurality of openings 1220 may comprise the same shape or a different shape.

The base 1210 may further comprise an engagement opening 1230 in a top and/or side of the base. The engagement opening 1230 may be configured to receive a user's hand (e.g., a finger), a handle, a hook, a rod, or other suitable engagement means so that the base may be picked up and moved. In this way, the base 1210 may be portable with or without deactivation containers contained therein. In some variations, the base 1210 may comprise a plurality of engagement openings (e.g., two, three, four, or more). The plurality of engagement openings may be on opposite sides of the base, such as the embodiment shown in FIG. 11.

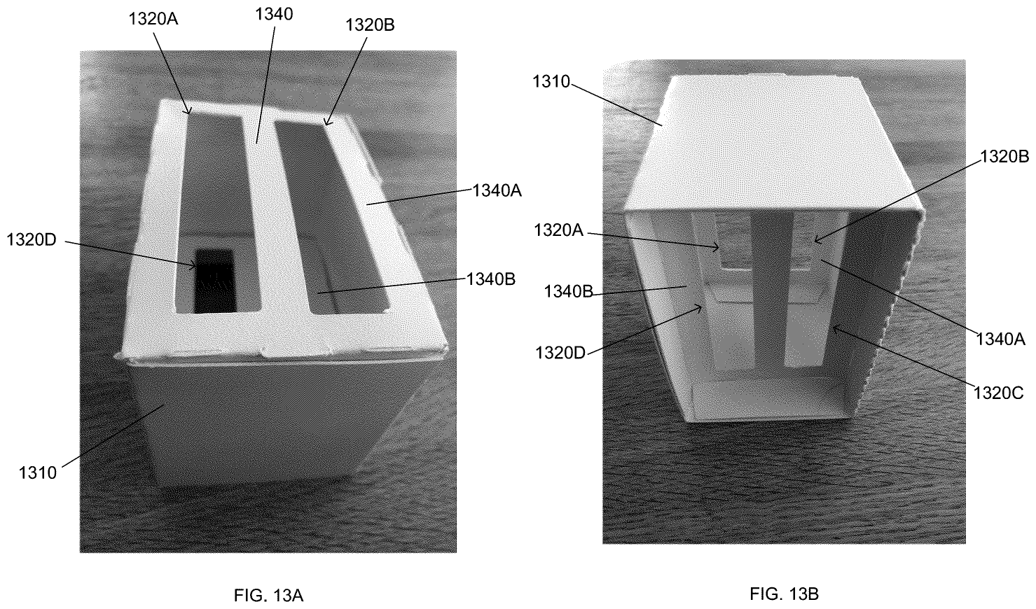

FIG. 13A and FIG. 13B show an illustrative variation of a base with an open bottom and upper and lower standoff members 1340. The standoff member 1340 has corresponding features to those described for FIG. 11. For example, a first standoff member 1340A may be at a first position and a second standoff member 1340B may be at a second position. The first position may be coplanar with a top edge of the base sidewall(s). The first standoff member 1340A may define openings 1320A and 1320B. The second position may be any position along a vertical dimension of the base 1310. The second standoff member 1340B may define openings 1320C and 1320D. As shown in FIG. 13B, the second position may be offset from a bottom of the base 1310. In this way, a deactivation container may be received by the base 1310 may be maintained in an upright position without tipping over.

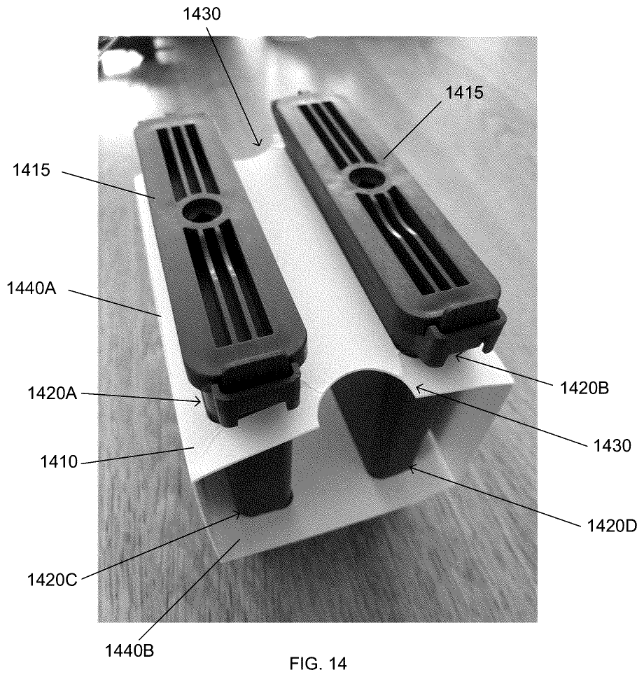

FIG. 14 shows an illustrative variation of a base with two standoff members connected by two sidewalls, no bottom, and with deactivation containers received therein. As illustrated, two sides of the base are open. The base 1410 may comprise an upper standoff member 1440A and a lower standoff member 1440B. The upper standoff member 1440A defines openings 1420A and 1420B, and the lower standoff member 1440B defines openings 1420C and 1420D. The openings 1420A-D may be similar to the openings 1120A-B described above with the respect to FIG. 11 and the openings 1220A-D described above with respect to FIG. 12, and may include any of the same features. As shown, the deactivation containers fit through the openings 1420C and 1420D such that the deactivation containers themselves (the bottom of the containers) contact a supporting surface, rather than the base itself.

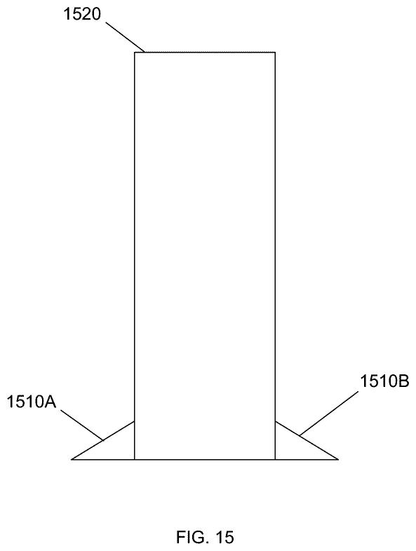

In some variations, the base may comprise one or more stability extensions. For example, FIG. 15 depicts an exemplary variation of a deactivation container and a base comprising stability extensions coupled to the deactivation container to assist in maintaining an upright position thereof. The base may comprise a plurality of stability extensions (e.g., two, three, four, five, six, seven, eight or more), such as a first stability extension 1510A positioned on a first side of the deactivation container 1520 and a second stability extension 1510B positioned on a second, opposite side of the deactivation container 1520. The stability extensions 1510A and 1510B may be integral with, or coupled to (releasably or fixedly), the deactivation container 1520 at or near a bottom of the deactivation container 1520. In this way, the extension may be in the form of feet that contact a surface upon which the deactivation container sits (e.g., a table, a countertop, or a clinical bench). For example, the stability extensions 1510A, 1510B may rest or may otherwise be releasably coupled to the surface upon which the deactivation container sits such that the stability extensions or feet prevent, or assist in preventing, the deactivation container from tipping over. In some variations, such as the variation depicted in FIG. 15, the stability extension(s) may be configured as a wedge (e.g., having a triangular cross-sectional shape) where a hypotenuse of the wedge extends from a sidewall of the container to the surface upon which the deactivation container sits. In other variations, the stability extension(s) may have a different shape. For example, the stability extension(s) may have a square, rectangular, semi-circular, semi-ovular, hexagonal, pentagonal, diamond, or other cross-sectional shape that allows for the stability extension(s) to be configured to counteract a force applied to a top of the deactivation container (e.g., with a flat contact surface). In some variations, the stability extension may be formed as a rectangular beam or hemisphere that protrudes from one or more external surfaces of the deactivation container. In such a variation, a flat contact surface of the rectangular beam or hemisphere may contact or sit flush again the surface upon which the deactivation container sits. In some variations, a plurality of sets of stability extensions 1510A, 1510B may be used and the sets of stability extensions may be spaced apart along the length of the deactivation container 1520.

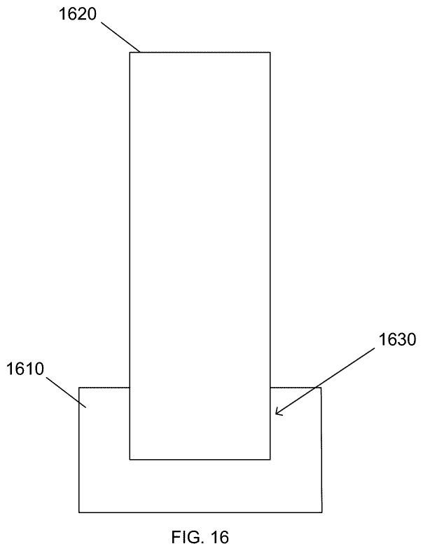

In some variations, the stability extension(s) may be configured to receive a portion of the deactivation container. For example, FIG. 16 depicts a base in the form of a stability extension 1610 configured to receive a deactivation container 1620. As shown there, the stability extension 1610 may be an elongate member (e.g., elongate beam) comprising a channel or groove 1630 configured to receive a portion of the deactivation container 1620 therein. For example, the stability extension 1610 may have a U-shaped cross-section. The stability member 1610 may extend from and/or along one or more (e.g., two) sidewalls of the container, and the bottom of the container 1620 may be in contact with a bottom surface of the channel or groove 1630. In this manner, the stability extension 1610 may be positioned under the deactivation container 1620 and between the deactivation container 1620 and the surface upon which the stability extension 1610 sits. In some instances, this may reduce or inhibit heat transfer between the container 1620 and the surface and/or may allow for easier transport of the container 1620 when coupled to the stability extension. The channel or groove 1630 may have a depth sufficient to engage at least a portion of the sidewalls of the container 1620. For example, the depth of the channel may be such that the container is maintained in an upright position when positioned within the channel or groove 1630. In some variations, the depth of the channel may correspond to the height of at least one sidewall of the deactivation container 1620, while in other variations, the depth of the channel may be less than the height of at least one sidewall. For example, in some variations, the depth of the channel may be about ⅕, about ¼, about ⅓, about ½, or about ⅔ the height of at least one sidewall of the deactivation container 1620. The length of the stability extension 1610 and/or the channel or groove 1630 of the stability extension 1610, may be greater than, equal to, or less than the length of the deactivation container 1620. In variations, in which the length of the stability extension 1610 is less than the length of the deactivation container 1620, the length of the stability extension 1610 may be about ⅕, about ¼, about ⅓, or about ½, or about ⅔ the length of the deactivation container 1620. In some variations, a plurality of stability extensions 1610 may be used and the stability extensions may be spaced apart along the length of the deactivation container 1620.

### g. Device

The deactivation kits/systems described herein may be configured to receive a device comprising a material for which deactivation or neutralization is desirable. These devices may be medical or non-medical devices. For example, in some variations, the devices may be configured to be used in a medical or cosmetic procedure, after which the device may be placed in the deactivation container. The devices may be configured to be placed on or otherwise contact skin to treat a patient. The device may comprise a pen, a wipe, a patch, or any other form suitable to contact a patient's skin.

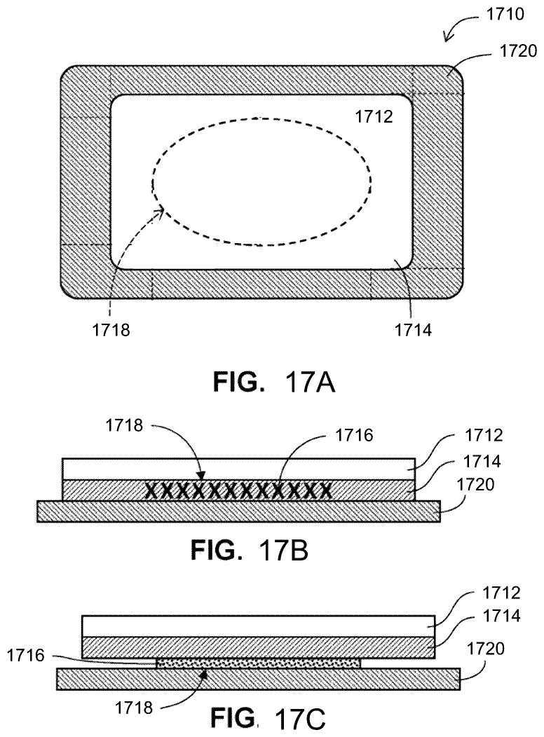

In an exemplary variation, shown in FIGS. 17A-C, the device may comprise a patch 1710 configured to be placed on a patient's skin to treat a condition. The patch may comprise a shape such as a rectangle, as shown, but in some variations may comprise a square, circle, triangle, or any other shape. The shape of the patch may be determined by a target treatment site. For example, the patch 1710 may be shaped to correspond to a palm, a finger, a hand, an underarm, a sole of a foot, or a portion thereof. The patch 1710 may comprise a planar substrate and may itself be substantially planar. The patch 1710 may comprise one or more layers such as, for example, a backing layer 1712, an adhesive layer 1714, an alkali metal 1716, and a release liner 1720. The number of layers and a thickness of each layer may determine the overall thickness of the patch.

The backing layer 1712 may be configured to provide a structure to which the alkali metal 1717 and/or adhesive layer 1714 may be applied. The backing layer 1712 may be unreactive with a patient's skin such that the backing layer may be handled (i.e., contacted) even if the person handling the device's skin is wet. The backing layer 1712 may comprise one or more of a fabric, a paper, a non-woven, a foil, a foam, a plastic film, or a polymeric film. The adhesive layer 1714 may directly or indirectly contact the backing layer. The adhesive layer 1714 may be dispersed or extended across a portion of the backing layer. The adhesive used in the adhesive layer 1714 may be safe for contact with human skin. The adhesive layer 1714 may be configured to be easily removed after a medical or cosmetic treatment. The alkali metal 1716 may be dispersed or extended across the adhesive layer 1714 and/or backing layer 1712. The area of the adhesive layer and/or backing layer over which the alkali metal layer is dispersed or extended may define a treatment region 1718 (which may correspond to a target treatment site of a user). The adhesive layer 1714 may be a peripheral adhesive around a perimeter of the treatment region. In some variations, the adhesive layer 1714 may be a continuous layer within which the alkali metal particles are laminated or embedded. The release liner 1720 may be applied over the alkali metal 1717 such that it contacts the adhesive layer 1714. The release liner 1720 may have the same dimensions as the treatment region 1718. The release liner may be impermeable to water so as to avoid any premature reaction between the alkali metal of the alkali metal layer and water in an external environment.

The alkali metal 1716 may comprise a single, free alkali metal that is substantially pure or may be an alloy or oxide. For example, the alkali metal may comprise neat sodium or neat potassium. In some variations, the alkali metal may comprise an alloy such as a sodium alloy, a potassium alloy, or a sodium potassium alloy. In further variations, the alkali metal may comprise an oxide. For example, the alkali metal may comprise sodium oxide (e.g., sodium monoxide, sodium peroxide, and sodium superoxide), a potassium oxide, or a sodium potassium oxide. The alkali metal may be a liquid, a solid, a semi-solid, or a combination thereof at room temperature (e.g., about 25 degrees C.). The alkali metal 1716 may form an alkali metal layer of a uniform thickness. In some variations, the thickness of alkali metal may vary across the alkali metal layer.

The alkali metal 1716 of the alkali metal layer may be configured to generate heat. More specifically, the alkali metal may be configured to exothermically react with water (e.g., moisture, sweat) such that an amount of heat is generated. The amount of heat generated in the exothermic reaction may depend on an amount of alkali metal in the alkali metal layer and/or an amount of water at or near a treatment site. In some variations, the treatment site may be dry (i.e., a zero or near-zero amount of water present at, within, or near the treatment site). The amount of heat generated may be useful in addressing a medical or cosmetic concern, such as in reducing, inhibiting, or minimizing excessive sweat. In some variations, the amount of heat generated may be useful in treating hyperhidrosis. The amount of heat generated may also be useful in inhibiting bacteria growth, and thus in decreasing odor that may be associated with bacteria growth. The amount of heat generated may render the treatment site and/or adjacent areas aseptic. The amount of heat generated may also be useful in treating a cosmetic condition or in treating an open wound.

### h. Alkali Metal Reactant or Alkali Metal Solubilizer

The deactivation kit described herein may include a fluid configured to react with a device. In an exemplary variation, the fluid may comprise a liquid configured to react with substantially all of the previously unreacted alkali metal of an alkali metal device contained within a cavity of a deactivation container. The liquid may be contained within the cavity of the deactivation container of the deactivation kit described herein. In some variations, a volume of liquid contained within the cavity may be about 0.5 L to about 2 L, such as about 1 L. The liquid may comprise an alkali metal reactant or alkali metal solubilizer, which may be a solution of one or more of an alcohol, a glycol, and water. The alcohol may be ethanol, isopropanol, t-butanol, stearyl alcohol, or tris(trimethylsilyl)methanol and the glycol may be propylene glycol. In an exemplary variation, the liquid may comprise propylene glycol with a volume of about 0.05 L to about 2 L, such as about 0.120 L. The propylene glycol may be mixed with at least water to form the volume of liquid described herein.

The reaction between the alkali metal of the alkali metal device and the alkali metal reactant or the alkali metal solubilizer may be exothermic. The exothermic reaction may increase a temperature of the deactivation container and/or the base. Utilizing one or more of the features described herein, such as the alignment members, dimensions of the deactivation container, dimensions of the alkali metal device, volume of fluid, pressure relief valves, and/or amount of alkali metal of the alkali metal device, external surfaces of each component of the deactivation container (e.g., each sidewall, the bottom, and the lid) may be maintained at or below a predetermined threshold temperature during and/or after the chemical reaction. The predetermined threshold temperature may be about 50 degrees C. during the reaction. This may advantageously allow a user to handle one or more of the deactivation container and base before, during, and/or after the chemical reaction and to prevent the reaction rate from increasing.

A duration of the chemical reaction may correspond to the time required for the alkali metal reactant or solubilizer to react with substantially all of the previously-unreacted alkali metal of the alkali metal device during the deactivation process described herein. The duration of the deactivation process may be referred to as the predetermined deactivation period. The predetermined deactivation period may correspond to a time required to completely react all previously unreacted alkali metal of the alkali metal device. The alkali metal device may be contained within the deactivation container for a storage period. The storage period may be longer than the predetermined deactivation period. For example, the alkali metal device may be contained within the deactivation container after the chemical reaction is substantially complete. In another example, the alkali metal device may be inserted into the deactivation container before the alkali metal reactant or solubilizer is inserted (e.g., poured) into the deactivation container. The predetermined deactivation period may be determined by one or more of an amount of unreacted alkali metal of the alkali metal device, a size of the alkali metal device, a concentration of alkali metal reactant or alkali metal solubilizer within the liquid contained with the deactivation container, a concentration of an additive combined with the liquid, and a volume of the liquid including the alkali metal reactant or solubilizer in the deactivation container during the deactivation process. In some variations, the predetermined deactivation period may be about 10 minutes to about 10 hours, about 30 minutes to about 8 hours, about 30 minutes to about 10 hours, or about 1 hour to about 8 hours.

The solution described above may further comprise one or more additives (e.g., buffer or acid) configured to maintain a pH of the solution below a predetermined pH level during and after the reaction, which may advantageously allow for safe (i.e., non-hazardous) handling and/or disposal of the deactivation container in the base and/or biohazard waste bag. The predetermined pH level may be a level considered hazardous for a human user. For example, the predetermined pH level may be between about 2.5 and about 12.5. In some variations, the lower limit of the predetermined pH level may be 1, 2, 3, 4, 5, or 6. In other variations, the upper limit of the predetermined pH level may be 7, 8, 9, 10, 11, or 12. In this way, the solution may not be hazardous for a user even in the event of a spill, a leak, and/or direct contact between the user and the solution. The additive may comprise one or more of an acid (e.g., citric acid, acetic acid, glycolic acid, azelaic acid, and salicylic acid) and a buffer (e.g., Trisisooctylamine with its hydrochloride salt, Triphenylacetic acid with its sodium salt, and tetrabutylammonium [N4444][HEPES]). The additive may be soluble in the alcohol, glycol, and/or water described herein.

### i. Biohazard Waste Bag

The deactivation kits described herein may further comprise a disposable container. The disposable container may be configured to receive one or more deactivation containers. In some variations, the disposable container may be configured to receive one or more bases coupled to, or separated from, deactivation containers. For example, the disposable container may be configured to receive a base containing one or more deactivation containers therein. In this way, the disposable container may be safe for disposal even when one or more deactivation containers (which may contain a device and/or a fluid) are contained therein. The disposable container may be configured to receive the one or more deactivation containers before, during, and/or after a deactivation process within each of the deactivation containers. The disposable container may be made of material(s) that are substantially impermeable to water and/or air, and may be elastic to accommodate more than deactivation container. Accordingly, the material of the disposable container may be polyethylene, polypropylene, or polyolefin material. The disposable container may be substantially impermeable to water and/or air.

In an exemplary variation, the disposable container may be a biohazard waste bag. The biohazard waste bag may comprise a resealable opening. The biohazard waste bag may have a cavity therein with a volume configured to receive two or more deactivation containers. In another variation, the disposable container may comprise a cardboard box. The cardboard box may comprise four sidewalls, with each sidewall connected such that the container maintains its fluid impermeability. For example, each sidewall may be connected via an adhesive (e.g., glue), a tape a sliding lock, or combination thereof in order to create a fluidic seal therebetween.

In some variations, two or more of the devices of the kits/systems described herein may be packaged together in common packaging. For example, one or more deactivation containers (e.g., one, two, three, four, or more), alkali metal reactant or alkali metal solubilizer (which may be within the cavity of the deactivation container or may be packaged separately from the deactivation container (e.g., in a syringe, cuvette, tubing, or other container) and one or more bases (e.g., one, two, three, four or more) may be provided together as a single or multi-use kit. For example, in some variations, the kit may include, within common packaging, two deactivation containers housing alkali metal reactant or alkali metal solubilizer therein, and one base. In this variation, the kit may be configured for use with one patient having two treatment areas (e.g., both underarms) or may be configured for use with two patients having one treatment area each. In some variations, one or more devices (e.g., alkali metal devices), such as two, three, four, or more, and/or one or more biohazard waste bags may also be provided within the common packaging.

### II. Methods of Deactivation

Also described herein are methods related to deactivating a device comprising an alkali metal after use, such as, for example, in a method of treatment. The treatment may be directed to skin injuries, skin wounds, skin conditions, hyperhidrosis (e.g., excessive sweating), undesirable bacteria growth on a human body, or other medical or cosmetic conditions. The alkali metal device may be configured to be applied to a site on a human body such that the alkali metal of the alkali metal device may exothermically react with a substance (e.g., water) on or within tissue of a treatment site. After the alkali metal device has been used, the remaining alkali metal of the alkali metal device may be deactivated and disposed of. Deactivation may occur in a deactivation process. The deactivation process may ensure that the alkali metal device may be safely disposed. The deactivation process may occur within a deactivation container via an exothermic reaction between an alkali metal reactant or alkali metal solubilizer contained therein and the remaining alkali metal of the alkali metal device.

The exothermic reaction between the alkali metal reactant and remaining alkali metal of the alkali metal device may result in one or more of a temperature increase of a sidewall of the deactivation container body, a pressure increase within the cavity of the deactivation container, generation of a gaseous byproduct, and a temperature increase of a sidewall of the deactivation container base. The deactivation methods described herein may address each potential hazard such that the deactivation process is safe and effective at deactivating any remaining alkali metal of the alkali metal device. Accordingly, as described above, the base of the deactivation kit may securely maintain the deactivation container in an upright position before, during, and/or after the deactivation process such that a valve (e.g., pressure relief valve) on or within the lid of the deactivation container may safely release a gaseous byproduct of the alkali metal reaction without spilling or leaking the alkali metal reactant or solubilizer. Additionally, the base and deactivation container may each comprise features configured to minimize the temperature increase of sidewalls thereof, such that a user (e.g., a patient, a researcher, or a clinician) may safely handle each component. Additionally, the deactivation container may include a security mechanism releasably coupled to the lid such that the valve is maintained in a closed configuration until opening is desired. The security mechanism may advantageously enable the deactivation container to be transported and/or moved without spilling or leaking any alkali metal reactant or solubilizer contained therein.

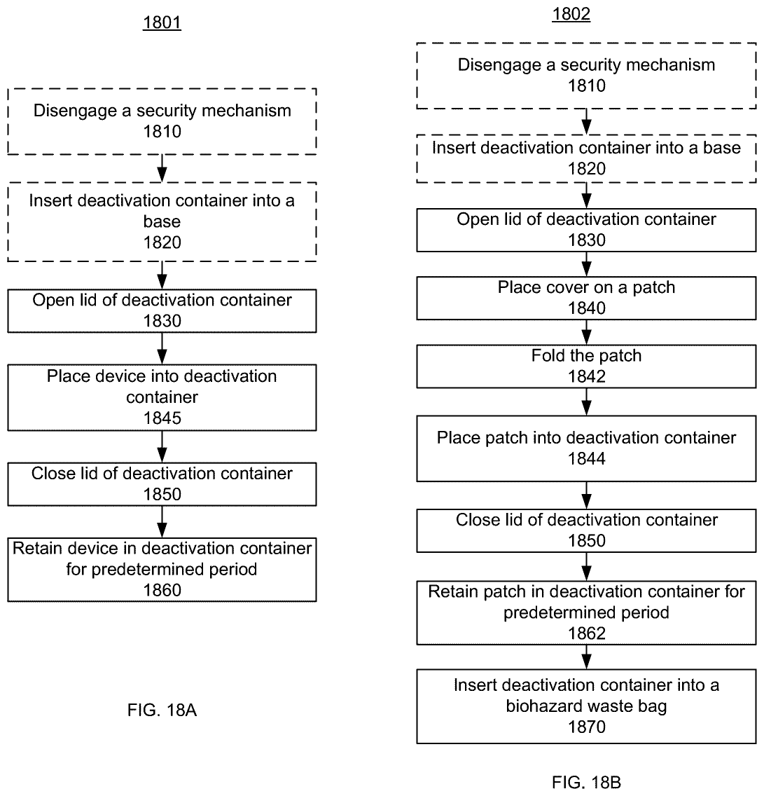

Methods of deactivating an alkali metal device may include deactivating the device after use of the device in a medical or cosmetic treatment. FIGS. 18A-B provide flowcharts of illustrative methods of deactivating a device using a deactivation kit. As shown in FIG. 18A, a method 1801 may comprise disengaging a security mechanism in a step 1810. Disengaging the security mechanism may comprise sliding a clip off of the lid and optionally the body, tearing a first perforation and a second perforation to separate a second portion of the security mechanism from a first and/or third portion and thereby release the security mechanism from the lid and optionally body and/or removing and/or releasing one or more of a latch, a magnet, a hook, an elastic band, and a cable tie. The security mechanism, when engaged or otherwise coupled to the container, may be configured to maintain the valve in a closed position. Accordingly, disengaging the security mechanism may enable the valve to transition to an open position, such as upon a pressure differential meeting or exceeding a predetermine pressure differential threshold. The deactivation container may be inserted into or otherwise coupled to a base. In some variations, the deactivation container may be provided to a user within and/or coupled to the base, such that the user needs to place or otherwise couple the deactivation container with or to the base. As described in detail herein, in some variations, the base may comprise an opening configured to receive the deactivation container and inserting the deactivation container into the base may comprise inserting the deactivation container into the opening (e.g., an opening of a standoff member). Once received within or otherwise coupled to the base, the deactivation container may be more easily maintained in an upright position and/or a stability of the deactivation container in the upright position may be increased.

In a step, 1830, the lid of the deactivation container may be opened. A device, such as an alkali metal patch, may then be placed in a cavity of the deactivation container in a step 1845. The deactivation container may contain a volume of fluid, such as an alkali metal reactant or alkali metal solubilizer. In some variations, the deactivation container may be provided with the fluid already contained therein (e.g., in the cavity), while in other variations, methods may include transferring the fluid (e.g., from a storage container such as a syringe or other container) into the cavity of the deactivation container. Thus, inserting the device into the deactivation may partially or completely submerge the device in the volume of fluid such that the unreacted alkali metal of the device contacts the fluid. Then, the lid of the deactivation container may be closed in a step 1850. The lid may be closed via the hinge such that a gasket of the lid engages a perimeter of an opening of the deactivation container. In some variations, closing the lid may comprise engaging a latch, such that an end of the lid opposite the hinge is releasably coupled to the deactivation container. Once closed, the lid may form a fluid-tight seal of the opening of the deactivation container.

The device may be retained in the deactivation container for a predetermined deactivation period in a step 1860, during which the deactivation process occurs. The predetermined deactivation period may be about 10 minutes to about 8 hours. A sidewall temperature of the deactivation container may be maintained at less than about 50 degrees C. during the predetermined deactivation period. A gaseous byproduct generated by the reaction between the alkali metal and the alkali metal reactant or solubilizer during the predetermined deactivation period may vent through the pressure relief valve of the lid, after a pressure within the deactivation container meets or exceeds the predetermine pressure differential threshold. In this way, the device may be deactivated and disposed of in a safe, efficient, and secure manner.

As shown in FIG. 18B, a method 1802 may comprise deactivating a device comprising an alkali metal patch. Similarly to the method described with respect to FIG. 18A, the method 1802 may comprise disengaging a security mechanism in step 1810, inserting or otherwise coupling the deactivation container to the base in step 1820 (if not already disposed within/coupled thereto), and opening the lid of the deactivation container in step 1830. In some variations, the device to be deactivated may comprise a patch with a layer of alkali metal, a backing layer, and a layer of adhesive. In these variations, it may be desirable to place a cover on the patch to partially or completely cover the layer of adhesive to prevent adhesive particulates from disengaging from the device and subsequently circulating within the fluid. Accordingly, in a step 1840, a cover may be placed on the patch (e.g., on a layer of patch adhesive). The cover may comprise a layer of porous material (e.g., paper, fabric), plastic film, polymeric film, or any other suitable material. Additionally, or alternatively, it may be desirable to fold the patch prior to placement within the deactivation container to ensure either adequate exposure of the unreacted alkali metal with the alkali metal reactant or solubilizer (e.g., folding tissue contacting surface outward) or to control the exposure of the unreacted alkali metal with the reactant or solubilizer (e.g., folding tissue contacting surface inward). Thus, in some variations, the method may further comprise folding the patch at step 1842. The patch may be folded such that a first portion of the backside of the patch (opposite the cover) contacts a second portion of the backside of the patch. In some variations, the patch may be folded more than once.

The patch may then be placed in a cavity of the deactivation container, in a step 1844, such that an alignment feature within the deactivation container maintains a gap between the patch and a sidewall and/or bottom of the deactivation container. Similar to method 1801, the lid of the deactivation container may be closed in a step 1850 and the patch retained in the deactivation container for a predetermined deactivation period in a step 1862. The deactivation container may then optionally be inserted into a biohazard waste bag in a step 1870 before disposing. The deactivation container may or may not be coupled to a base when inserted into the biohazard waste bag and/or disposed of. While described above as containing certain steps, it should be understood that the methods of deactivation may include any subset of deactivation steps in any suitable order. For example, in some variations, the deactivation container may be provided to the user already coupled to the base. In another example, the deactivation container may be placed in the biohazard waste bag before inserting the device into the deactivation container (while keeping the biohazard waste bag open so that gas may flow unconstrained from the deactivation container to an external environment). In yet another example, an alkali metal device (such as the patch described herein) may be removed from the deactivation container upon expiration of the predetermined deactivation period (i.e., all previously unreacted alkali metal thereof has been safely deactivated) and placed directly into the biohazard waste bag.

All references cited are herein incorporated by reference in their entirety.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

While embodiments of the present invention have been shown and described herein, those skilled in the art will understand that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-30. (canceled)

31. A deactivation container for a device comprising an alkali metal, the deactivation container comprising:
a container body defining a cavity therein configured to receive the device, wherein the cavity contains an alkali metal reactant or an alkali metal solubilizer therein; and
a lid coupled to the container body and partially received in the cavity in a closed position, wherein the lid comprises a pressure relief mechanism to release gas from a top of the deactivation container during a deactivation process.

32. The deactivation container of claim 31, wherein the lid further comprises at least one indentation.

33. The deactivation container of claim 31, wherein the lid is coupled to the container body by at least one hinge.

34. The deactivation container of claim 33, wherein the hinge is coupled to a support coupled to the container body.

35. The deactivation container of claim 35, wherein the support is integrally formed with or fixedly attached to the container body.

36. The deactivation container of claim 33, wherein the at least one hinge comprises a stop that prevents the lid from opening beyond a predetermined angle.

37. The deactivation container of claim 31, wherein the predetermined angle is between about 90 degrees to about 180 degrees.

38. The deactivation container of claim 31, wherein the lid comprises a latch.

39. The deactivation container of claim 31, wherein the alkali metal reactant or the alkali metal solubilizer comprises a solution comprising one or more of ethanol, isopropanol, t-butanol, stearyl alcohol, tris(trimethylsilyl)methanol, and propylene glycol.

40. The deactivation container of claim 31, wherein the deactivation container further comprises at least one alignment feature within the cavity.

41. The deactivation container of claim 31, wherein the pressure management feature comprises a vent with a lumen extending transversely through the lid.

42. The deactivation container of claim 41, wherein the lid further comprises:
a recess on a bottom of the lid at least partially surrounding the vent; and
a membrane within the recess and configured to limit fluid exchange between the cavity of the deactivation container and an external environment via the vent.

43. The deactivation container of claim 42, wherein a diameter of the recess is greater than a diameter of the vent.

44. The deactivation container of claim 43, wherein the diameter of the vent is about 0.01 mm to about 2 mm.

45. The deactivation container of claim 42, wherein the membrane has a pressure rating of about 5 psi to about 8 psi.

46. The deactivation container of claim 31, wherein a thickness of the lid increases from both of a first end and a second end of the lid to a central portion of the lid.

47. The deactivation container of claim 31, wherein the lid further comprises a plurality of longitudinal ridges configured to increase a stiffness of the lid.

48. The deactivation container of claim 47, wherein the plurality of ridges is on a top surface of the lid.

49. The deactivation container of claim 31, wherein the lid further comprises one or more projections extending from a bottom surface of the lid and configured to contact the device within the deactivation container when the lid is in the closed position.

50. The deactivation container of claim 31, wherein the pressure management feature comprises a valve extending through the lid and at least partially into the cavity of the container body.

51.-91. (canceled)
